(19) United States
(12) Patent Application Publication
(10) Pub. No.: US 2015/0050311 A1
(43) Pub. Date: Feb. 19, 2015

(54) NOVEL METHOD FOR THE PREPARATION OF A STRAIN-ADAPTED VACCINE

(71) Applicant: Ludwig-Maximilians-Universitat München, München (DE)

(72) Inventors: Sören Schubert, München (DE); Andreas Wieser, München (DE)

(73) Assignee: Ludwig-Maximilians-Universitat München, München (DE)

(21) Appl. No.: 14/383,487

(22) PCT Filed: Apr. 5, 2013
(86) PCT No.: PCT/EP2013/057216

§ 371 Date: Sep. 5, 2014

(30) Foreign Application Priority Data
Apr. 5, 2012 (EP) 12163468.7

(51) Int. Cl. A61K 39/02; A61K 39/112; A61K 39/108

(52) U.S. Cl. CPC A61K 39/0291; A61K 39/0258; A61K 39/0266; A61K 39/0275
USPC 424/190.1; 536/23.4; 435/69.3; 514/44 R

(57) ABSTRACT

The present invention relates to a method for the preparation of a strain-adapted vaccine specific for a bacterial strain, comprising the steps of: (a) genetically engineering a bacterial strain obtained from a subject, wherein said genetic engineering comprises introducing a nucleic acid molecule encoding a fusion protein, wherein the fusion protein comprises a bacterial membrane protein fused to at least one affinity tag, (b) growing the genetically engineered bacterial strain obtained in step (a) in solution, (c) isolating membrane vesicles from the growth culture of step (b) by affinity purification using the affinity tag, and (d) formulating the membrane vesicles isolated in step (c) into a strain-adapted vaccine. The present invention further relates to a nucleic acid molecule encoding a fusion protein comprising a bacterial membrane protein fused to at least one affinity tag and a kit comprising said fusion protein.

### CROSS REFERENCE TO RELATED APPLICATION(S)

This application is a National Stage application under 35 U.S.C. §371 of PCT/EP2013/057216, filed Apr. 5, 2013, which claims the benefit of priority of EP 12163468.7, filed Apr. 5, 2012, the entire content of each of which is incorporated herein by reference.

### BACKGROUND OF THE INVENTION

Infections by multi-resistant bacteria are more and more common and have developed into a major health concern in many facilities around the world. Not only are these opportunistic pathogens colonisers of patients and health care staff, they also cause infections which are difficult if not impossible to cure with common antibiotic regimens. Therefore, these pathogens are responsible for a significant loss of patient lives (Ho et al. 2010; O'Fallon et al. 2009).

In some cases, patients infected with these pathogens form immune responses which protect against the infection caused by this individual strain. This is typically observed in fully immuno-competent humans who do not get ill upon infection, or in patients that survived an infection with untreatable bacteria for a prolonged period of time because their immune system has mounted effective reactions against targets in the pathogenic organisms. To date, no vaccines are available that provide protection from infection or colonisation resistance against these types of bacteria in those patients that are not immuno-competent or capable of mounting an appropriate defence. Whole bacterial vaccines which are based on either attenuated live bacteria, or dead bacteria, have the drawback that they are difficult to produce and certify for safety and immunogenicity. In those cases where they are replicative but attenuated and produce no toxin or virulence factors, they are most often mere colonizers. The immune system recognizes colonizers as such and does not mount an efficient immune response against those. However, in order to produce a sufficient immune response, they need to stimulate the immune system with “danger-signals”. Whereas invasion is one of the most effective danger signals, it also puts every patient at risk of systemic infection. Although it can theoretically be envisaged to evoke an immune response to multi-resistant organisms via the application of inactivated bacteria together with immunogenic substances, it would need to be assured that the risk for the patient to actually become infected is as low as possible. Accordingly, the bacteria must be inactivated effectively. The drawback of such inactivation, however, is most often a change in the immunogenic structures, especially the conformation epitopes, as they are easily altered by heat or chemicals/detergents/alcohols etc. Furthermore, detrimental substances inherited in bacteria which can for example cause shock and immunologic hyperreactions, can still be present and active. For example endotoxin (LPS) will be released from the bacteria during manipulation and is not inactivated by heat or most chemical substances, thus representing a further drawback of this potential approach.

The individual strains of a particular bacterial species often differ considerably in their ability to produce polysaccharide capsules and other features, resulting in different antigenic profiles between different strains. Accordingly, the number of known conserved antigens expressed by all or at least the majority of strains of a particular pathogen species is limited, thereby hindering the development of globalised, strain-independent vaccines. Also, the use of purified epitopes for vaccination does not overcome this problem, as individual differences in human immune receptors will prevent individual patients from recognising the desired epitopes. In view of the lack of suitable global vaccines, it would be desirable to provide a method for the quick preparation of vaccines that are specific for a particular problematic bacterial strain isolated in a ward of interest or from a patient of interest, i.e. an index patient, in order to protect other patients or future patients against this particular strain. Similar considerations also apply to e.g. farms, animal shelters as well as other animal accommodations, in which protection of animals from a specific strain present in said farm, shelter or accommodation is desirable. A quick preparation is desirable, as it reduces the spread of the pathogen and can prevent outbreaks, thereby reducing the loss of lives as well as the economic burden imposed by treating affected patients.

Membrane vesicles formed by bacteria, such as e.g. outer membrane vesicles (OMVs) formed by Gram-negative bacteria but also membrane vesicles (MVs) of Gram-positive bacteria, have been found to be immunogenic and are, therefore, a promising tool for vaccine preparations.

OMVs are formed from fragments of the outer membrane of Gram-negative bacteria and have an average diameter of 10 to 300 nm. OMVs are often shed by Gram-negative bacteria into their environment and typically comprise outer membrane proteins, lipids, phospholipids, periplasmic and cytoplasmic proteins as well as lipopolysaccharides.

Haneberg et al. 1998 as well as Bakke et al. 2001 describe nasal vaccines consisting of outer membrane vesicles from group B Neisseria meningitides, capable of inducing both local mucosal as well as systemic antibody responses. Comparison with intramuscular injection (Haneberg et al. 1998) or with subcutaneous injection (Bakke et al. 2001) revealed a largely similar pattern of serum antibody specificities against the OMV components. However, in secretions only the nasal vaccine resulted in the development of antibody responses. The OMVs were prepared in both works by extraction of the bacteria with deoxycholate and subsequent differential centrifugation for the separation of OMVs.

WO03/051379 describes OMVs from Gram-negative bacteria and their use as vaccine compositions that provide broad spectrum protection from a number of bacterial strains and at least a wide range of strains within a singly bacterial genus. To this end, the OMVs are derived from a diversity of bacterial sources. Further described are modified OMVs that present additional antigens of interest on their surface or that lack the expression of certain antigens on their surface.

Holst et al. 2009 reviewed the properties and clinical performance of vaccines containing OMVs from Neisseria meningitides. The authors stress the problem that outbreaks of bacterial infections are often dominated by a specific strain present at one particular location, thus hindering the preparation of a universal vaccine. The authors predict that the OMV-concept will be further expanded and that a number of cross-protective antigens will be included in the vaccines, thereby potentially fulfilling the desire to develop a global vaccine strategy that enables susceptible individuals to be protected against all the relevant strains or serogroups of the respective pathogen.

The suitability of OMVs as vaccines has further been investigated by Bin et al. 2010, who showed the vaccine efficacy of OMVs from bacteria pathogenic for fish, namely the bacterium Edwardsiella tarda.

Chen et al. 2010 describe the adjuvant properties of OMVs when engineered with foreign antigens. By fusing the exogenous antigen of interest to a native bacterial protein known to be enriched in OMVs, a strong response in immunised mice was obtained even for antigens that are poorly immunogenic when administered as a purified antigen, such as e.g. GFP. Accordingly, this method provides a tool for preparing a vaccine based on (a) known antigen(s). As is emphasised by the authors, one important advantage of this method of producing recombinant OMVs carrying the exogenous protein lies in the vesicle purification by facile ultracentrifugation, which sidesteps the complex purification process of traditional subunit vaccines.

More recently it has been shown that also Gram-positive bacteria shed membrane vesicles (MVs). For example, Rivera and colleagues describe extracellular vesicle production by Bacillus anthracis (Rivera et al. 2010). These membrane vesicles were found to be double-membrane spheres with a diameter between 50 and 150 nm. Moreover, the vesicles were found to be immunogenic in BALB/c mice, which produced an IgM response to the toxin components present in the membrane vesicles. Mice immunised with these vesicles lived significantly longer than control mice. In addition, Gurung et al. 2011 describe the production of membrane vesicles by S. aureus during in vivo infection and show that these vesicles play a role in the delivery of bacterial effector molecules to host cells. The authors found that despite the structural differences between OMVs and MVs, they nonetheless shared many morphological characteristics. The authors of these two publications further pointed out that additional reports have described the release of membrane vesicles by the Gram-positive bacteria Staphylococcus aureus, Mycobacterium ulcerans, Bacillus spp., B. cereus and B. subtilis.

Despite the fact that a lot of effort has been invested into methods of providing vaccines against multi-resistant bacteria, no method exists so far to that is sufficiently specific and results in the provision of a vaccine within a short time frame, in order to avoid unnecessary losses of lives. Moreover, methods currently employed for the purification of membrane vesicles are mainly based on ultracentrifugation, which requires an excess of 100.000×g for extended periods of time. As a consequence, membrane vesicles can accumulate, fuse and may loose their natural properties when pelletted in such centrifugation steps. Moreover, ultracentrifuges are rather costly to purchase and operate and are generally only available in highly specialised centres or production facilities. In addiction, the rotors employed in ultracentrifuges most often cannot accommodate large volumes, as they would result in balancing problems and, therefore, only small volumes can be handled at one time. Accordingly, there is still a need to provide highly specific and fast methods for the preparation of vaccines against pathogenic bacteria.

This need is addressed by the provision of the embodiments characterised in the claims.

### SUMMARY OF THE INVENTION

The present invention relates to a method for the preparation of a strain-adapted vaccine specific for a bacterial strain, comprising the steps of: (a) genetically engineering a bacterial strain obtained from a subject, wherein said genetic engineering comprises introducing a nucleic acid molecule encoding a fusion protein, wherein the fusion protein comprises a bacterial membrane protein fused to at least one affinity tag, (b) growing the genetically engineered bacterial strain obtained in step (a) in solution, (c) isolating membrane vesicles from the growth culture of step (b) by affinity purification using the affinity tag, and (d) formulating the membrane vesicles isolated in step (c) into a strain-adapted vaccine. The present invention further relates to a nucleic acid molecule encoding a fusion protein comprising a bacterial membrane protein fused to at least one affinity tag and a kit comprising said fusion protein.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### BRIEF DESCRIPTION OF THE DRAWINGS

FIG. 1—(A) Antibody response against OMV based vaccine in the mouse model. Western-Blot analysis against concentrated protein (1: DHFR-m45) as a control and purified OMV (2). The vaccine doses were given on days 0 and 5, each 100 per nostril. IgM response was analysed after seven days, IgG after 14 days; serum-dilution was 1:1000, secondary antibody was used according to the manufacturers instructions. (Anti-IgM, Anti-IgG-HRPO-conjugate, sigma Aldrich); (B) Antibody response (IgG) against the OMV vaccine (a) and bacterial lysate of the whole strain (b). Serum taken at 14 days after immunization start, dilution 1:1000, secondary antibody against mouse IgG was used according to the manufacturers instructions. R: Fermentas PageRuler™pre-stained standard.

FIG. 2—(A) An avian pathogenic E. coli (APEC) OMV vaccine given i.m. to chicken (Lohmann LSL) without further adjuvating substances; 50 μl was delivered on days 0 and 7. Western-Blots were performed on days 0, 7 and 21; A significant Titre increase (IgY) to more than 1:500 can be shown after seven days of vaccination. (B) Analysis of the immune response after oculo-nasal application of OMV 50 μl on days 0 and 7. Western-Blots were performed on days 0, 7 and 21; significant Titre increase (IgY) to more than 1:500 can be shown after seven days.

FIG. 3—Western blot analysis of serum of a patient infected with pan-resistant Klebsiella pneumoniae for more than eight months with sepsis, osteomyelitis and pneumonia. Survival for this period suggests a protective immune response. Whole cell lysates and OMV vaccine preparations were subjected to one dimensional SDS PAGE and subsequent blot transfer onto nitrocellulose membranes. After o/n blocking with 3% skimmed milk, patient's serum (with all inherent antibodies) was applied at a dilution of 1:5000 (3% BSA PBS) for 1 h at room temperature. Bound serum Antibodies were detected with the secondary antibody Anti Human-IgG-HRPO-conjugate (developed in goat) from sigma Aldrich diluted in 3% BSA PBS 1:10.000. Visualization was performed with ECL subtrate (Pierce) according to the manufacturers instructions. (A): equal protein concentrations of OMV (1) and whole cell lysate (2) were used (see Coomassie stained right lanes). The Western-Blot revealed a strong immune-response against the OMV vaccine formulation and the organism itself, although this organism bears a capsule (left lanes). (B): When the OMV vaccine is diluted below the limit of detection by regular SDS Gel and Bradford protein measurement (1, right lanes) the Western Blot signal remains strong against the vaccine formulation proving high specificity and titres against the vaccine formulation (left lanes); Antigens seem to be enriched in the vesicles compared to the whole cell lysate. R: Fermentas PageRuler™pre-stained standard.

FIG. 4—Schematic gene map of the “hypersecretion”-product. The construct is under the control of the rpsM ribosomal protein promotor which is always active during bacterial growth. The leader sequence promotes export of the protein into the periplasm, the TolR homologue blocks interaction in the Tol-Pal system leading to hypervesiculation. The rrnB terminator ends mRNA synthesis.

FIG. 5—Schematic representation of the mode of action of the “hypersecretion”-product. The construct (false ligand) is exported through the inner membrane (IM) into the periplasmic space (PS). There it interacts with parts of the tol-pal system or its homologues and leads to enhanced OMV production. (OM=outer membrane; OMV=outer membrane vesicle, OMP=outer membrane proteins).

FIG. 6—Schematic gene map of the “purification”-construct used. The operon is under control of the rpsM ribosomal protein promotor which is always active during bacterial growth. The leader sequence promotes export of the protein into the periplasm and is cleaved thereafter. The strep-one tag integrated after the first linker sequence is displayed on the bacterial outside and used for interaction with the purification column. Between the strep-one double strep tag and the second linker, a multiple cloning site has been included to express further antigens on the outside of the bacterial cell for other purposes, if desired. The stalk domains raise the purification domains above the level of LPS and other proteins of the bacterial surface. With the trimerisation signal, the protein will insert its beta sheets into the membrane and trimerize forming the whole complex with a transmembrane anchor. The total length of the gene is 822 bp, which code for 273 aa residues.

FIG. 7—(B) Schematic of the insertion of the gene into the membrane of the bacteria and OMV. Proteins are inserted to form trimers and display the purification domains above the membrane level. (A) Western Blot against the strep-one tag (StrepMAB-Classic HRP Conjugated™; 1:2000) of whole cell lysate of a plasmid negative strain (1) and a plasmid positive strain (2). Monomers (cytoplasmic) have the size of about 25 kDa, Trimers are about 100 kDa and cannot be separated even by boiling in SDS solution.

FIG. 8—Flow chart of the ultra-centrifugation-free affinity column purification process. Liquid cultures are grown to produce sufficient amounts of OMV, bacterial cells are removed by sterile filtration (A) optionally enhanced by low speed centrifugation. Sterile supernatant is filtered trough a strep tag binding colomn (Strep-Tactin® Superflow®) according to the manufacturer's instructions (C). The tagged OMV will bind to the column, the column is run in gravity flow or vacuum mode. Flow through of excess media and OMV above the binding capacity of the column is discarded (D). Western Blot of plasmid negative strain (1) and two isolates with plasmid (2, 3) show strong expression of the purification product. Detection as described in FIG. 7.

FIG. 9—Flow chart of elution of the OMV fraction from the column loaded as described in FIG. 8. The whole cell pellet (D 1) is removed by filtration and the supernatant is applied to the column (A). Concentrated supernatant (1 ml dried and resuspended for SDS gel) shows presence of the purification tag on OMV (D 2). The flow through contains traces of unbound OMVs (1 ml concentrated on lane D 3). The column can be washed to remove debris and contaminations. A 1 ml concentrated wash solution shows no signal (D 4). Elution is dose dependent and strongest in the fractions 2 and 3 (according to the manufacturer's protocol). A small portion of pooled eluate 2 and 3 demonstrates a strong signal of the trimer (100 kDa) in lane D 5.

FIG. 10—Exemplary representation of one of the plasmid constructions employed herein, harbouring both an OMV hypersecretion inducing- (TorA-TolR) and purification enabling (Oca-purification) gene locus. Promotors as well as origin (p15A) are capable of working in multiple species (broad host range). Chloramphenicol resistance allows for selection also in multi-resistant organisms.

FIG. 11—Example of other bacterial organisms as described in Example IV (Salmonella enterica, Klebsiella pneumoniae, Yersinia enterocolitica, four different isolates used for each strain). Western Blot analysis shows strong expression of the purification product as well as trimerization. Insertion into the membrane is present when trimerization occurs. This can also be visualized with fluorescence microscopy. StrepMAB-Immo Antibody was labelled with Alexa fluor 488 (Thermo Scientific DyLight 488) and used on dried bacteria without fixation (Yersinia (A), Klebsiella (B), Salmonella (C)). Oca +: bacteria with the plasmid, Oca −: Wild type bacteria.

FIG. 12—Secondary loading of antigens coupled to a tag ligand sequence onto tagged vesicles by simple mixing protocol.

### DETAILED DESCRIPTION OF THE INVENTION

Accordingly, the present invention relates to a method for the preparation of a strain-adapted vaccine specific for a bacterial strain, comprising the steps of: (a) genetically engineering a bacterial strain obtained from a subject, wherein said genetic engineering comprises introducing a nucleic acid molecule encoding a fusion protein, wherein the fusion protein comprises a bacterial membrane protein fused to at least one affinity tag, (b) growing the genetically engineered bacterial strain obtained in step (a) in solution, (c) isolating membrane vesicles from the growth culture of step (b) by affinity purification using the affinity tag, and (d) formulating the membrane vesicles isolated in step (c) into a strain-adapted vaccine.

The term “vaccine”, as used herein, is defined in accordance with the pertinent art and relates to a composition that induces or enhances immunity of an individual to a particular disease. To this end, the vaccine comprises a compound that is similar to the pathogen or a compound of said pathogen causing said disease. Upon contact with this compound, the immune system of the individual is triggered to recognise the compound as foreign and to destroy it. The immune system subsequently remembers the contact with this compound, so that at a later contact with the disease-causing pathogen an easy and efficient recognition and destruction of the pathogen is ensured. In accordance with the present invention, the vaccine may be in any formulation for vaccines known in the art, such as for example mucosal vaccines, vaccines for intramuscular injection or vaccines for subcutaneous or intradermal injection as well as vaccines for inhalation, such as e.g. as aerosols. Such vaccine formulations are well known in the art and have been described, e.g. in Neutra M R et al. 2006 Mucosal vaccines: the promise and the challenge 6(2):148-58 or F. P. Nijkamp, Michael J. Parnham 2011; Principles of Immunopharmacology ISBN-13: 978-3034601351

Most preferably, the method of the present invention is for preparing a strain-adapted, mucosal vaccine for immunisation through e.g. oral, nasal, rectal or vaginal routes. The mucous membranes cover the aerodigestive and the urogenital tracts but also the eye conjunctiva, the inner ear and the ducts of all exocrine glands. They possess mechanical and chemical cleaning mechanisms that degrade and fend off pathogens and other foreign substances. In addition, a highly specialised innate and adaptive mucosal immune system protects these surfaces, and thus the body, against potential threats from the environment. Non-limiting examples of mucosal vaccines include nasal sprays, nose or eye drops as well as rectal or vaginal gel formulations.

In accordance with the present invention, the term “strain-adapted vaccine” relates to a vaccine that is specific for one individual strain of a bacterial species. Accordingly, the vaccine stimulates immunity towards this one particular bacterial strain. The term “vaccine specific for a bacterial strain”, as used herein, relates to a vaccine that immunizes against the bacterial strain of interest, but does not or essentially does not immunize against other bacterial strains of the same species nor to bacteria of other species. The term “a vaccine that essentially does not immunize against other bacterial strains of the same species nor to bacteria of other species”, as used herein, refers to a vaccine that immunizes against the bacterial strain of interest with at least 2-times higher efficiency than against a different bacterial strain or species, more preferably with at least 5-times higher efficiency, such as e.g. at least 10-times higher efficiency, more preferably with at least 50-times higher efficiency, and even more preferably with at least 100-times higher efficiency.

Also the term “bacterial strain” is used herein in accordance with the pertinent prior art and relates to a genetic variant, subtype, O-Antigen type or lysotype of a bacterial species. Typically, bacterial strains evolve in a localised manner, such as a hospital ward or an animal farm. However, due to e.g. contacts between patients or doctors with people outside said ward or due to e.g. the sale of animals to other farms, the bacterial strain may be distributed more widely, such as e.g. observed in pandemics.

The term “genetically engineering”, as used herein, refers to the process of bringing into a bacterial cell nucleic acid sequences that are not present in said bacterial strain prior to the step of genetic engineering, thereby modifying the genetic information of the bacteria. This is generally accomplished by transfecting or transforming a bacteria with the nucleic acid molecule, for example by electroporation, chemotransformation, cationic lipid mediated transfection, phage mediated transduction, conjugation, infection or other methods. Such methods are described in many standard laboratory manuals, such as Sambrook et al.; Molecular Cloning: A Laboratory Manual; Cold Spring Harbor Laboratory Press, 2nd edition 1989 and 3rd edition 2001.

The newly introduced nucleic acid sequence(s) may be incorporated into (a) chromosome(s) of the bacteria or may be present as extra-chromosomal sequences; both options are explicitly encompassed by the terms “genetically engineering” and modification of the genome of a bacterial strain. In accordance with the present invention, it is preferred that the nucleic acid molecule encoding the fusion protein is not incorporated into the genome of the bacteria but remains extra-chromosomal.

In accordance with the present invention, the term “nucleic acid molecules”, also referred to as nucleic acid sequences herein, includes DNA, such as cDNA or genomic DNA, and RNA. It is understood that the term “RNA” as used herein comprises all forms of RNA including mRNA, ncRNA (non-coding RNA), tRNA and rRNA. The term “non-coding RNA” includes siRNA (small interfering RNA), miRNA (micro RNA), rasiRNA (repeat associated RNA), and other RNA molecules that interfere with regulation, transcription or translation in bacteria. Both, single-strand as well as double-strand nucleic acid sequences are encompassed by this term. Further included are nucleic acid mimicking molecules known in the art such as synthetic or semi-synthetic derivatives of DNA or RNA and mixed polymers, both sense and antisense strands. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA) and locked nucleic acid (LNA) (see Braasch and Corey (2001) Chem. Biol. 8, 1). LNA is an RNA derivative in which the ribose ring is constrained by a methylene linkage between the 2′-oxygen and the 4′-carbon. They may contain additional non-natural or derivatised nucleotide bases, as will be readily appreciated by those skilled in the art.

It will be appreciated that for the modification of the genome of a bacterial strain, the nucleic acid molecule can be combined, preferably in a vector, with additional elements, such as e.g. regulatory elements but also sequences encoding selectable marker genes, i.e. markers which confer a selectable trait, such as an antibiotic resistance to the organism they are expressed in. Further additional elements may e.g. be fluorescent molecules or molecules which add specific metabolic traits to the organism they are expressed in, such as the fluorescent molecules or molecules adding specific metabolic traits described in more detail herein below.

Regulatory elements are required to ensure expression of the encoded protein as well as replication of the nucleic acid molecule in particular in those cases where the newly introduced nucleic acid molecule is present as an extra-chromosomal sequence or where regulation via endogenous regulatory elements is not ensured. Non-limiting examples of regulatory elements include an origin of replication, a promoter as well as terminating sequences. Additional regulatory elements may include translational enhancers and translation initiation codons, Shine-Dalgarno boxes or internal ribosomal entry sites or signal sequences capable of directing the expressed fusion protein to a cellular compartment, such as e.g. the plasma membrane, the outer membrane or the periplasm.

It is preferable, that the origin of replication can work in a broad range of host bacteria and does not produce an excess of copies in order not to affect the host organism too much. Suitable origins of replication (ori) include, for example, the p15A, Col E1, M 13, pBR 322, RK2, pUC18, RSF1010, RK 404 or pLAFR5 origins of replication as well as their derivatives. Most preferably, the on is of broad host range such as the p15A, RK 404 or pLAFR5 origins of replication (Chang, A. C. Y. and Cohen, S. N. Construction and characterization of amplifiable multicopy DNA cloning vehicles derived from the P15a cryptic miniplasmid, J. bacterial., 134, 1141-1156, 1978).

The promoter initiates transcription of the nucleic acid sequence and can be used to control the level of gene expression, while the terminator sequence ends transcription. Preferably, the promoter should be highly active. It is further preferred that the promoter is a native and conserved promoter that is independent of inducing systems, such as e.g. IPTG or arabinose, in particular in those cases where the respective bacteria do not have the necessary properties. Non-limiting examples of promoters include the lacZ promoter, ribosomal protein promoters such as rpsM promoter, antibiotic resistance gene promoters, such as the β-lactamase or chloramphenicol or inducible AHT resistance promoters, promoters of metabolic genes or the hcp promoter, the lac, trp or tac promoter, the lacUV5 promoter, as well as the T7, SP6 or T3 promoter.

Most preferably, the promoter is the rpsM ribosomal protein promoter (Hautefort I et al., Single-copy green fluorescent protein gene fusions allow accurate measurement of Salmonella gene expression in vitro and during infection of mammalian cells. Appl Environ Microbiol. 2003 December; 69(12):7480-91), which is highly conserved among different bacteria and provides the further advantage of being highly active and being activated whenever the bacteria perform metabolic processes.

Non-limiting examples for regulatory elements ensuring transcription termination include rho dependent and independent terminators such as the rrnB terminator, as well as phage derived terminators. Terminators can be included downstream of the nucleic acid sequence of the invention.

Most preferably, the terminator is at least partly factor independent based on inverted repeats such as the rrnB terminator, lambda tR2, trpR or hupB (Snyder and Champness, Molecular genetics of bacteria, ASM Press, third ed.; ISBN-13: 978-1-55581-399-4).

Signal sequences capable of directing the expressed fusion protein to a cellular compartment should preferably also be highly conserved. In accordance with the present invention, it is preferred that the signal sequence directs the expression of the fusion protein such that the portion of the fusion protein comprising the affinity tag is located to the outside of the membrane of Gram-positive bacteria or to the outside of the outer membrane of Gram-negative bacteria. Signal sequences are used to direct the protein to the site of membrane insertion or/and to help during insertion in case this process is not autocatalytical.

The skilled person is aware of means and methods of determining which part of a membrane protein is located to the outside of a bacterial membrane such as unspecific biotinylation of the native bacteria and determination of the biotinylated stretches of the respective protein, as biotinylation only takes place outside the bacterial membrane. Alternatively, all extracellular protein parts can be digested with aggressive peptidase/protease and the transmembranous or intracellular parts of the proteins can be retrieved and analysed in order to determine to which protein they belong.

For the Oca proteins employed in the appended examples it is known that the N-terminus of the protein is located to the outside of the bacteria. Accordingly, the affinity tag is coupled to the N-terminus of the Oca proteins in the examples employed herein.

To make sure that the modified proteins indeed fold in a way that the tag is presented to the outside of the membrane, various approaches are known in the art. For example, a loop protein could be applied inbetween two transmembrane stretches. As such loops are often less elastic or altered in their secondary structure, the correct representation should be confirmed after alteration of the protein. Preferably, the tag(s) is/are located and covalently bound to the N- or C-terminal end of the protein extending freely into the outside space of the bacteria.

Non-limiting examples of signal sequences are e.g. the Oca-leader sequence for localisation to the outer membrane or specialized Sec- or Tat-leader sequences for localisation to the periplasm, as described e.g. in Robinson et al. Transport and proofreading of proteins by the twin arginine translocation (Tat) system in bacteria; Biochimica et Biophysica acta 1808 (2011) 876-884 or Thien B. Cao et al. The general protein secretory pathway: phylogenetic analyses leading to evolutionary conclusions; Biochimica et Biophysica acta 1609 (2003) 115-125.

Selectable markers enable to determine which cells are transformed with the nucleic acid sequence. Suitable markers are known in the art and include, without being limiting, resistance genes for the antibiotics chloramphenicol, G418, neomycin or kanamycin, ampicillin, hygromycin, polymyxin B, tetracycline or their derivates or chinolones.

Preferably, as the clinically relevant pathogenic strains of particular relevance in accordance with the present invention often show resistance to a wide variety of different antibiotic resistance traits, a resistance gene is employed for an antibiotic that is active in most of the pathogenic strains of relevance, i.e. has no intrinsic resistance. Even more preferably, the antibiotic is no longer used clinically, thus ensuring a low resistance rate in the patient isolates, i.e. the lack of an acquired resistance even in pan-resistant organisms.

Alternatively to the use of antibiotic resistance markers, plasmid addiction systems, also referred to as toxin-antitoxin systems (such as PhD-Doc, Hok-Sok, ccdB, MazEF, RelBE, or Restriction-modification systems) can be applied to ensure plasmid stability during growth. Selection can be performed with insertion of metabolic traits (such as lacZ etc.) or dyes such as fluorescent dyes (e.g. GFP, YFP, CFP, mcherry, dTomato, mOrange, sapphire, mPlum etc and derivates thereof). Those markers can be applied if it is more convenient than resistance markers.

Preferably, the vector is a plasmid, cosmid, virus, bacteriophage or another vector used e.g. conventionally in genetic engineering. The nucleic acid molecule employed in genetical engineering in accordance with the present invention may be inserted into any one of several commercially available vectors. Non-limiting examples include plasmid vectors such as the pUC-series, pBluescript (Stratagene), the pET-series of expression vectors (Novagen) or pCRTOPO (Invitrogen), pBR 322 derivatives, pACYC184 derivatives including pMCL derivatives, RSF1010, RK 404, pLAFR5 and their derivatives.

The nucleic acid sequences inserted in the vector can e.g. be synthesized by standard methods, or isolated from natural sources or produced semi-synthetically, i.e. by combining chemical synthesis and recombinant techniques. Ligation of the coding sequences to transcriptional regulatory elements and/or to other amino acid encoding sequences can be carried out using established methods, such restriction digests, ligations and molecular cloning. For vector modification techniques see for example Sambrook and Russel “Molecular Cloning, A Laboratory Manual”, Cold Spring Harbor Laboratory, N.Y. (2001).

In accordance with the method of the present invention, the genetic engineering comprises introducing a nucleic acid molecule encoding a fusion protein, wherein the fusion protein comprises a bacterial membrane protein fused to at least one affinity tag.

The resulting fusion protein thus is a protein comprising at least two protein subunits, namely one for a bacterial membrane protein and a second one for at least one affinity tag, in a single amino acid chain which does not naturally occur as a single amino acid chain.

The subunits may be connected, i.e. fused, directly to each other or indirectly via (a) peptide sequence(s) that is/are not naturally part of the sequence of either subunit. Such additional peptide sequences include, without being limiting, sequences which may be included in order to avoid sterical hindrance between the subunits, thereby enhancing the correct functional formation of the subunits of the fusion protein. Further additional peptide sequences include sequences suitable to present the affinity tag(s) more advantageously, such as described herein below with regard to the “stem domain” as well as sequences facilitating the correct incorporation/attachment of the fusion protein into/onto the membrane. Additional peptide sequences may, where necessary, also serve as helix breakers, for example when comprising prolins, thus avoiding the formation of c′-helices. Most preferably, the fusion protein comprising a bacterial membrane protein fused to at least one affinity tag and encoded by the nucleic acid molecule employed in the method of the invention comprises at least one such additional peptide sequences. Also preferred is that the nucleic acid molecule encoding the fusion protein is prepared by ligation of the nucleic acid sequences encoding the respective subunits and, optionally, the additional peptide sequence(s), thereby forming a single coding region comprising the coding regions for both subunits (and optionally the additional sequence(s)) in a single un-interrupted reading frame.

The term “bacterial membrane protein”, as used herein, is defined in accordance with the pertinent prior art and relates to a protein that is incorporated into, attached to or firmly associated with the membrane of a bacterium. Bacterial membrane proteins include, without being limiting integral membrane proteins, which are permanently bound to the lipid bilayer, peripheral membrane proteins that are temporarily associated with the lipid bilayer or with integral membrane proteins and lipid-anchored proteins that are bound to the lipid bilayer through lipidated amino acid residues. In addition, pore-forming toxins and many antibacterial peptides, although being water-soluble molecules, can undergo a conformational transition upon association with lipid bilayers and, consequently, can become reversibly or irreversibly membrane-associated. Preferably, at least the functional part of the membrane protein is oriented towards the outside of the bacterial cell and even more preferably protrudes above the naturally occurring membrane parts such as lipopolysaccharides (LPS) in gram negative bacteria, thereby ensuring accessibility.

Preferably, the protein is a protein incorporated into, attached to or firmly associated with the cytoplasmic lipid membrane of Gram-positive bacteria or the outer membrane of Gram-negative bacteria. Most preferably, the bacterial membrane protein is a protein incorporated into the cytoplasmic lipid membrane of Gram-positive bacteria or the outer membrane of Gram-negative bacteria. Particularly preferred examples of bacterial membrane proteins are provided herein below.

The term “affinity tag”, as used herein, is defined in accordance with the pertinent art and relates to protein or peptide tags that are attached to proteins in order to enable purification based on the binding of said tag to an interacting moiety, such as a natural interaction partner as is the case for the chitin binding protein or the maltose binding protein, or to a matrix such as a metal matrix as is the case for the poly(His) tag, which binds to nickel-containing affinity media. Due to the binding of the tag to the interacting moiety, the tag-comprising protein, and cellular structures associated therewith, can be purified from a sample.

Non-limiting examples of affinity tags include Strep-tags, chitin binding proteins (CBP), maltose binding proteins (MBP), glutathione-S-transferase (GST), FLAG-tags, HA-tags, Myc-tags, poly(H is)-tags as well as derivatives thereof. All these tags as well as derivatives thereof are well known in the art and have been described, for example in Lichty J J et al. Comparison of affinity tags for protein purification Protein Expr Purif. 2005 May; 41 (1): 98-105. The Strep-tag family of tags is purified using streptavidin or strep-tactin tetrameric protein complexes (Schmidt and Skerra, Nature protocols 2007 The Strep-tag system for one-step purification and high-affinity detection or capturing of proteins DOI: 10.1038/nprot.2007.209). Chitin binding proteins (CBP) also allow native purification with automatic cleavage and removal of the tag sequence. The binding partner-matrix is chitin (offered for example by NEB). Maltose binding proteins bind to amylose coated matrices. Glutathione-S-transferase (GST) as a tag binds to glutathione labelled matrices. The FLAG, MYC and HA tags bind to respective monoclonal antibodies coupled to a matrix. The His-Tags form complexes with nickel or cobalt-ions also bound by the purification matrix.

In a preferred embodiment, the affinity tag is not an epitope tag. Epitope tags are short peptide sequences capable of binding to high-affinity antibodies, such as for example the V5-tag, c-myc-tag and the HA-tag.

In another preferred embodiment, the affinity tag is chosen such that the affinity purification is possible under non-denaturing conditions. For example, using a Strep-tag, which elutes under gentle, physiological conditions, it is possible to isolate membrane vesicles without destroying the function of the proteins present on these membrane vesicles. Alternatively, the membrane vesicles may be eluted by enzymatic cleavage of the affinity tag from the membrane protein. This approach also avoids destruction of surface proteins present on the membrane vesicles by using enzymes of high specificity for the cleavage site used, such as e.g. a factor X enzyme derived from the human blood coagulation cascade.

The term “at least one”, as used herein, encompasses also at least two, such as at least three, at least four or at least five or more, such as at least six, at least seven, at least eight, at least nine, at least ten or even at least 20. It will be appreciated by the skilled person that this term further encompasses exactly one, exactly two, exactly three, exactly four, exactly five, exactly six, exactly seven, exactly eight, exactly nine, exactly ten or exactly 20. In accordance with the present invention, the terms “one”, “two”, “three” etc., as opposed to “at least one”, “at least two”, “at least three” etc. are restricted to referring to exactly one, two, three etc.

With regard to the affinity tag, it is particularly preferred that at least two affinity tags are present in the fusion protein. The inclusion of a higher number of affinity tags results in an improved, more stable binding to the moiety employed for affinity purification.

It is further preferred that the fusion protein comprises further amino acid sequences as described herein above that enable the presentation of the affinity tag on the surface of the membrane vesicle such that it extends further away from the surface than the remaining molecules present on the surface of the membrane vesicles, such as e.g. LPS or capsule material, such as e.g. structures that are normally part of or associated with the bacterial capsule, usually sugar moieties.

Accordingly, such an additional amino acid sequence also referred to herein as a “stem domain”, is incorporated into the fusion protein between the membrane protein and the affinity tag. As a result, the affinity tag is more easily accessible by the moiety employed for affinity purification, thereby facilitating purification of the membrane vesicles. A suitable amino acid sequence may be easily identified by the skilled person; generally, any amino acid sequence may be employed. A preferred example of such an additional amino acid sequence is shown in the examples below and is represented by SEQ ID NO:1. A nucleic acid sequence encoding the amino acid sequence of SEQ ID NO:1 is shown in SEQ ID NO:2.

It is further preferred that the fusion protein comprises additional sequences facilitating the correct incorporation/attachment of the fusion protein into/onto the membrane. For example, signal sequences capable of directing the expressed fusion protein to a cellular compartment as described herein above may be included to facilitate the correct transport of the fusion protein into e.g. the periplasmic space in Gram-negative bacteria. Examples of such signal sequences, also referred to as leader sequences herein, have been provided above and are shown in the appended examples. Preferably, a leader sequence is chosen that is cleaved off of the fusion protein after transport to the correct cellular compartment either autocatalytically or by highly conserved systems that are functional in many different bacterial species such as the Tat or Sec-systems described in more detail above.

Further, sequences needed for insertion into the membrane preferably are autocatalytically inserting sequences or are sequences that are able to use any highly conserved mechanisms present in many different species. The main conserved systems for protein insertion into the outer membrane are the YfiO, YfgL, NlpB (and their respective homologues such as Omp85) dependent pathways (also called YaeT or BamA-E complex). These systems utilize a protein complex comprising several partners to translocate and insert/fold membrane proteins of β-strand transmembrane domains in the bacterial outer membrane. The system is linked either to the periplasmic chaperone SurA or Skp (or their respective homologues in each species considered). For other proteins, the pathways are not as well established. However, chaperones such as e.g. DegP and FkpA seem to play a role in hindering premature folding and helping with efficient insertion into the outer membrane.

Autotransporter insertion into the membrane has been found to be to some extent autocatalytically and can be performed in vitro without any cofactors. In vivo the BamA system has been shown to enhance insertion at least for some autotransporters. However the YaeT or BamA-E system is present in all bacteria and is involved in good outer membrane function.

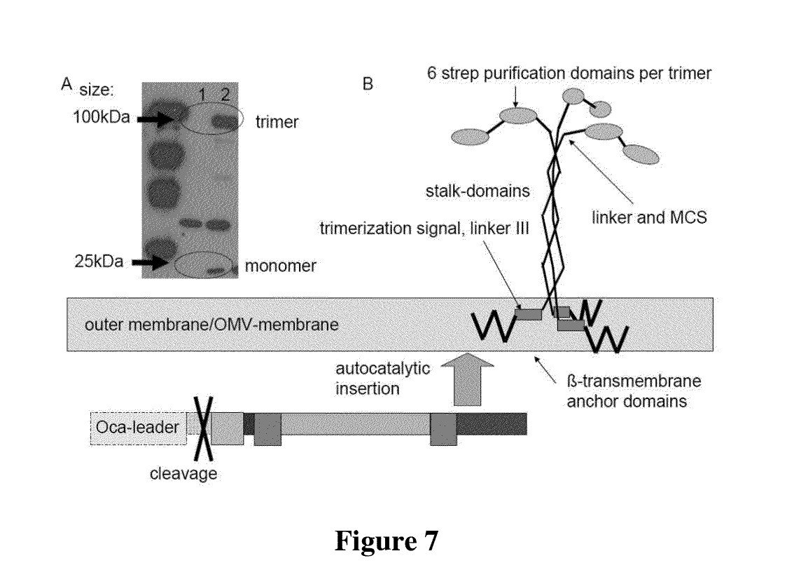

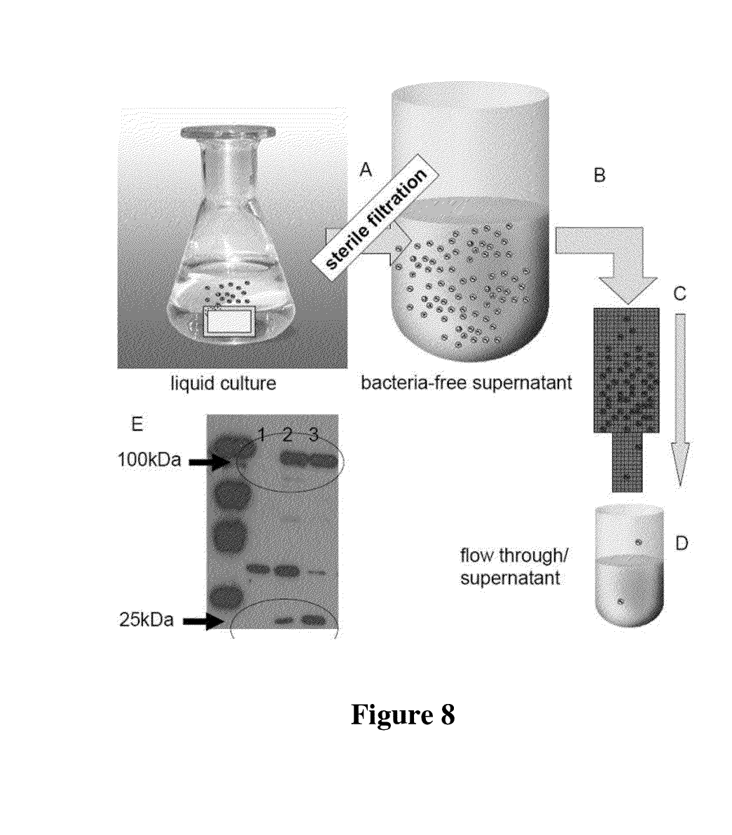

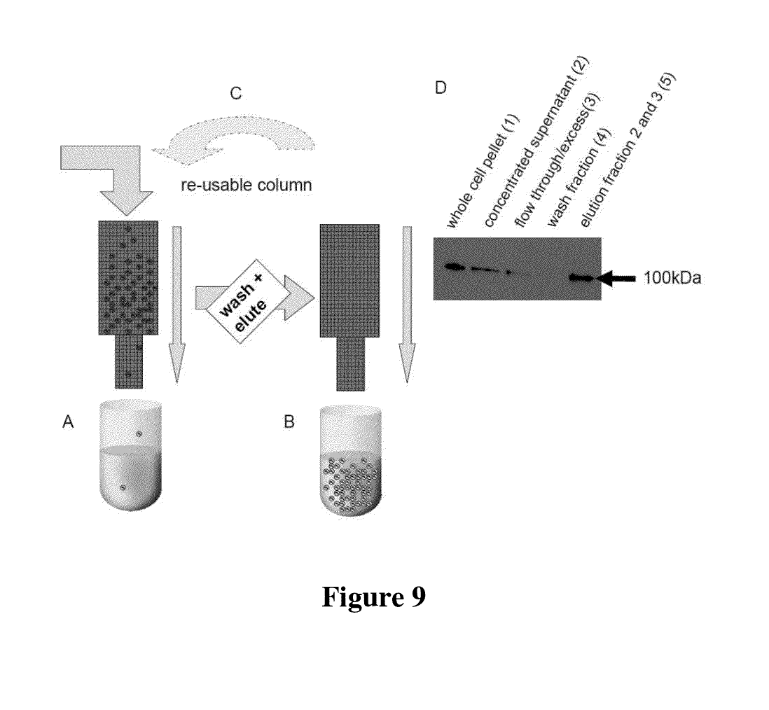

Most preferably, the protein used to develop the extracellular tag display in accordance with the present invention is a homologue of a trimeric autotransporter protein (as described e.g. in Cotter S E, Surana N K, St Geme J W 3rd. Trimeric autotransporters: a distinct subfamily of autotransporter proteins. Trends Microbiol. 2005 May; 13(5):199-205), wherein the trimerization domain is located between the stem and the transmembrane domains and wherein the formation of stable trimers is autocatalytically carried out (see e.g. FIG. 7B). Most preferably, a system is chosen that is autocatalytically active and can perform translocation across the inner membrane with a highly conserved system and insertion into the outer membrane on its own, as this improves the correct integration/attachment of the fusion protein independent of host proteins for processing, cleavage or insertion into the membrane. For example, in gram-negative bacteria, a leader sequence such as a Seq- or Tat-targeting sequence can be employed that transports the fusion protein into the periplasm, while insertion into the membrane and trimerisation is mediated by the Oca protein itself (see e.g. Gohlke et al. The TatA component of the twin-arginine protein transport system forms channel complexes of variable diameter. Proc Natl Acad Sci USA. 2005 Jul. 26; 102(30):10482-6; Pugsley A P. Translocation of proteins with signal sequences across membranes. Curr Opin Cell Biol. 1990; 2:609-616; Cotter S E, Surana N K, St Geme J W 3rd. Trimeric autotransporters: a distinct subfamily of autotransporter proteins. Trends Microbiol. 2005 May; 13(5):199-205).

In accordance with the present invention, the subject from which the bacterial strain has been obtained may be any human or animal subject susceptible to bacterial infection. For example, the subject may be selected from humans, companion animals such as cats, dogs or rabbits, farm animals such as horses, cows, pigs, deer, fish, chicken or other poultry, animals kept for example in zoological gardens such as e.g. primates and monkeys, wild cats, canines, elephants, bears, giraffes, marsupials etc. Preferably, the subject is a human subject or a farm animal and most preferably, the subject is a human subject.

In a further step of the method of the invention, the genetically engineered bacterial strain obtained in step (a) is subsequently grown in solution.

Suitable conditions for culturing bacteria in solution are well known to the person skilled in the art. For example, such conditions may including growing the bacteria under aeration in Luria Bertani (LB) medium, Frantz' medium or modified Catlin-6 medium (MC.6M), Columbia-Blood Agar, Choco Agar, Mueller Hinton Agar, XLD or MacConkey Agar, CIN Agar, AV Agar, etc. (Neumeister, Braun, Kimmig; Mikrobiologische Diagnostik Thieme Verlag ISBN-13: 978-3137436027). All these media and their composition are well known in the art. Alternatively, the bacteria may also be grown without aeration where appropriate. The skilled person is aware of how to choose appropriate agar concentrations to ensure that the medium remains a solution.

In addition, the medium can be buffered or supplemented with suitable additives known to enhance or facilitate growth of the respective bacterial species. For example, E. coli can be cultured from 4° C. to about 37° C. In general, the skilled person is also aware that these conditions may have to be adapted to the needs of the bacterial species under investigation. In those cases where an inducible promoter controls the expression of the nucleic acid molecule of the invention, expression can be induced by addition of an appropriate inducing agent.

Bacterial growth in solution is obtained by growing the cells in a liquid medium, preferably under agitation in an incubator/shaker at controlled temperatures. In addition, humidity and/or carbon dioxide content may be adjusted as required.

Growth in solution ensures that a sufficient amount of bacteria is obtained by the method of the invention, which is not practically possible when employing bacterial cultures on solid plates. One of the drawbacks of using bacterial cell culture plates is that they cannot be sufficiently closed, thus enhancing the contamination risk. In addition, during the process of harvesting bacteria from solid plates, traces of the solid plate material can be carried along, thereby contaminating the bacterial material as well as the membrane vesicle preparation used for purification. Such contaminations cannot be removed sufficiently by centrifugation or column purification and, thus, remain in the membrane vesicle preparation even after purification. A further drawback is that during bacterial culture of solid plates, an exact determination of cell growth is no possible, thus rendering it difficult to estimate the ideal time point for harvesting the bacteria. Moreover, preparing large amounts of bacteria-dependent growth solutions comprising the specific nutrients and supplements required for a particular organism is more easily achieved using liquid media than using solid media. Finally, liquid media provide the additional advantage of providing a better and more controllable bacterial growth.

Preferably, the amount of solution employed is at least 10 ml, more preferably at least 50 ml, such as e.g. at least 100 ml, at least 500 ml, at least 1 l and more preferably at least 5 l. Even more preferably, the amount of solution employed is at least 10 l.

It will be appreciated that the term “growing the genetically engineered bacterial strain obtained in step (a)” refers to growth of the bacterial strain after it has been subjected to genetical engineering. Depending on the efficiency of the genetical engineering approach chosen, this bacterial population obtained in (a) may comprise a mixture of bacteria, not all of which express the fusion protein. Accordingly, the term “genetically engineered bacterial strain” refers to a bacterial strain comprising at least 20% of bacteria that were successfully engineered with the respective nucleic acid molecule, more preferably at least 50%, such as e.g. at least 70%, such as e.g. at least 80% and more preferably at least 90%. More preferably, the bacterial strain comprises at least 95% of bacteria that were successfully engineered with the respective nucleic acid molecule, such as e.g. at least 98% and even more preferably at least 99%. Most preferably, all of the bacteria obtained in step (a) have been successfully engineered to express the fusion protein in accordance with the invention.

It will be appreciated that the percentage of successfully engineered bacteria may be increased by growing the bacteria obtained in step (a) in the presence of a selective agent, for which a resistance gene was incorporated in the nucleic acid molecule employed for genetic engineering. Accordingly, it is preferred that step (b) of the method of the invention is carried out in the presence of an antibiotic to which the successfully genetically engineered bacteria of step (a) are resistant. The transformed bacteria may also be selected by other means described previously and a positive clone can be singularized and chosen for all further procedures.

In accordance with a third step of the method of the invention, membrane vesicles are isolated from the growth culture of step (b). Preferably, the isolation of membrane vesicles is carried out when the bacteria have grown to a sufficient density, such as at least half the density of their stationary phase density, or as dense as their stationary phase density. More preferably, the optical density at 600 nm as measured with standardized protocols should be at least 0.5, more preferably above 0.8 and most preferably above 1.0.

Means and methods for determining the density of a bacterial culture are well known in the art and include, without being limiting, measurement of optical densities, real time PCR, photoluminescence, absorbance, flow cytometry etc. Depending on the bacteria cultured, cells may need to grow for at least 8 hours, more preferably at least 10 hours, such as e.g. at least 12 hours, in order to obtain a sufficient density.

As used herein, the term “membrane vesicles” is defined in accordance with the pertinent art and relates to spherical membrane-enclosed structures. Bacterial membrane vesicles have been described for both Gram-negative bacteria, i.e. outer membrane vesicles (OMVs), and Gram-positive bacteria, as discussed herein above.

Membrane vesicles are released from the bacterial membranes and accumulate in the supernatant of the bacterial culture.

Preferably, isolation of membrane vesicles comprises the steps (i) obtaining the supernatant from the bacterial culture after step (b), (ii) optionally filtering the supernatant and (iii) applying the supernatant or filtered supernatant to an affinity purification system, whereby the eluate of the affinity purification step comprises the isolated membrane vesicles. The filtration step (ii) may be employed to remove residual bacteria or infectious particles as well as large extracellular debris. Suitable filter pore sizes used typically range between 0.2 μm and 0.5 μm. Preferably, the filters applied should have low protein binding affinity in order to not remove the desired membrane particles. Non-limiting examples of such filters having low protein-binding affinity include PVDF, PES or polysulfone.

Affinity purification is well known in the art and relates to a purification method based on the highly specific interaction between two binding partners, such as e.g. a ligand and its receptor or an enzyme and its substrate. Generally, one of the binding partners is coupled to a solid phase or medium, typically a gel matrix, such as for example agarose. The second partner is usually present in a mixture from which it is to be purified. For the purification, the mixture containing the second partner is brought into contact with the solid phase or medium to which the first partner is bound. Due to the high binding specificity between the two binding partners, the second partner becomes entrapped, while all other molecules are not bound. The solid phase/medium is then removed from the mixture, washed and the target molecule released from the entrapment via elution.

Binding of the second partner to the first partner associated with the solid phase may be achieved by column chromatography, also referred to herein as affinity chromatography, whereby the solid medium is packed onto a column, then the mixture is allowed to flow through the column followed by a washing step in which a wash buffer is run through the column. Finally, the elution buffer is applied to the column and the purified second partner is collected. These steps are usually done at ambient pressure but may also be carried out under high pressure or under vacuum. Also known in the art are methods in which the liquid phase is pumped in from the bottom, is thus pushed through the solid phase and exits at the top of the columns. Alternatively, or additionally, binding may be achieved using a batch treatment, by adding the initial mixture to the solid phase in a vessel, mixing, separating the solid phase, for example by centrifugation or sedimentation, removing the liquid phase, washing, re-centrifuging or sedimentation, adding the elution buffer, re-centrifuging or sedimentation and removing the eluate. Affinity columns can be eluted by changing the ionic strength through a gradient, for example by changing salt concentrations, pH, pI, charge or ionic strength.

Preferably, elution of the bound particles is carried out under non-denaturing conditions as described herein above, thereby enabling the purification of native vesicles.

It will be appreciated that the affinity purification is a purification based on the interaction of the affinity tag(s) of the fusion protein with a binding partner. Preferred is that the affinity purification is not a antibody-antigen purification, as in some cases an immunogenic response may be triggered against the antibody used for purification of the membrane vesicles.

In the next step, the membrane vesicles obtained after affinity purification are formulated into a strain-adapted vaccine. Formulation into a vaccine may be achieved, for example, by mixing the vesicles with buffer solutions to stabilise the pH in the formulation. Non-limiting examples of such buffers include NaCl 0.9%, Tris, PBS or Ringer solution. Also preferred is that the buffers are independent of carbon dioxide levels. Such buffers include, for example, HEPES or MOPS. Further additives may include, without being limiting, adjuvating substances to enhance the immune response (as described herein below) or other chemical compounds to enhance stability and storage life such as sucrose.

It will be appreciated that the steps of the method of the invention are carried out in the order as recited herein, i.e. from (a) being the first step to (d) being the last step.

In accordance with the present invention, a method of obtaining strain-specific vaccines against pathogenic bacteria, including multi-resistant bacteria, within a short time frame is provided. Optimal protection against such (multi-resistant) bacteria is ideally achieved by offering the immune system a mix of antigens that are not denatured, fixed or altered, e.g. proteins in their native conformation, sugar moieties of capsules, lipids and lipoproteins of the outer membrane. This is of particular importance, as for example conformity epitope recognizing antibodies will only bind proteins in their native conformation. Accordingly, these native structures can evoke a balanced and neutralising immune response capable of providing colonisation resistance as well as protection in the infection model. So far, such responses can only be found in patients infected with the pathogen itself for long enough to develop protective immunity.

Membrane vesicles provide this mix of antigens, as they provide antigens in their native conformation and also present lipids and sugars of the capsule. Accordingly, they were shown recently to evoke a sufficient immune response (Alaniz et al. 2007). Furthermore, colonisation resistance against enterotoxigenic E. coli could be demonstrated recently by OMVs used as a vaccine in mice (Roy et al. 2011). However, membrane vesicles isolated from a random reference strain cannot provide protection against a specific problematic strain, as capsule antigens and surface composition vary considerably between individual strains. As discussed herein above, a lot of effort has been invested at developing globalised, strain-independent vaccines that are universally applicable to all strains of a particular pathogen. In accordance with the present invention, emphasis was placed instead on identifying a quick and easy to perform method of isolating membrane vesicles from a particular, problematic strain.

The bacterial membrane and the protein-, lipid- and sugar-complexes associated therewith are the main targets the body will react against in case of infections with pathogenic bacteria. Accordingly, providing the complex of the bacterial membrane (including capsule) to the immune system eliminates the need to identify conserved targets and optimising them for use in the appropriate genetic background of the human population, as is usually required with purified epitopes. Such optimisation processes are time consuming and expensive and require large resources. However, in cases of local outbreaks, time and resources are always in short supply and such optimisations are so far impossible to perform in the short timeframe available.

To efficiently use membrane vesicles as a vaccine, large amounts of membrane vesicles need to be produced quickly after isolation of a specific bacterial strain from a specific site/patient/animal. This is achieved by the method of the present invention, which is based on the introduction of a purification tag that allows the purification and washing of the membrane vesicles without the need for denaturing conditions and ultracentrifugation of large volumes at speeds exceeding 150,000×g for several hours.

As shown in the appended examples, an expression system can be developed that ensures that the affinity tag is located to the outside of the outer membrane above the LPS or capsule factors and is presented to the affinity resin/beads used. The system shown in the appended examples is a completely synthetic construct based on the theory of trimeric auto-transporter systems (Oca) (Cotter et al. 2005). The gene consists of a leader sequence which is automatically removed after passing the inner membrane. Insertion into the outer membrane and subsequent trimerisation is autocatalytic. The long stalk domain raises the affinity tags over the LPS and membrane proteins to improve access to the binding domains of the purification matrix without the problems of breaking off typically experienced with fimbrial adhesions. The latter further have the disadvantage that they cannot be found on all of the membrane vesicles of the various pathogenic species and even when they are present, they usually have an insufficient density to be functional for purification.

To allow fast transition into human medicine a derivate of the strep-tag was used which is already in use for cell purifications in human medicine.

Vaccinations act by stimulating the immune system of the host against specific antigens present on pathogenic organisms, so that a preformed immune response is elucidated. This can either prevent the infection by preventing the first steps of colonization of the organism (which normally takes place at mucosal surfaces) or by neutralizing and eliminating the organism or toxic products after invasion. Regular vaccination systems applied today mainly use purified proteins which are intramuscularly injected together with an adjuvating substance. This stimulates mainly the production of immunoglobulins of the subclass IgG and leads to immune-cell homing into the muscles and soft tissue.

Alternative methods such as denaturated pathogenic organisms or particles (such as inactivated viruses) are also used as vaccine together with adjuvating substances. This technique is in principle comparable to the above mentioned system using a purified antigen, but more different targets are available for the immune system as the whole organism is used for vaccination. However, these formulations need to be inactivated and thus have the same disadvantage as purified proteins, i.e they are often changed in their conformation. This can alter the properties of immunogenic determinants or even eliminate the originally present antigens. The most effective vaccination strategy is still the injection of attenuated pathogens, such as viruses which are still replicative but attenuated, so that they can be cleared by the immune system easily. This approach also leads to mainly IgG responses and homing of the immune cells to the muscles and soft tissue where the vaccine was injected/the organism is replicating. However, the antigens of the pathogen are all presented to the immune system in a natural, not denaturated state and the intrinsic immunostimulation is used for triggering the immune system. Inherent dangers of this method are the possibility of severe infections in immunocompromised hosts after vaccination (or the infection of such patients by vaccinated subjects) as well as the side effects by the immune response such as joint pain and fever.

In addition to the drawback recited above, all classical ways of (human) vaccination do not sufficiently stimulate the mucosal immune response to protect against colonization with pathogenic organisms.

The vaccine of the present invention combines the positive features of the known vaccine systems while avoiding their disadvantageous properties and at the same time further adds ease of administration and production. The vesicle based vaccines can for example be administered on the mucosal surfaces and will naturally interact with them and the local as well as systemic immune system. The vesicles further comprise a wide variety of antigens and inherent immunostimulatory substances found on the bacterial surface comparable to the attenuated vaccines used for viral disease. However, as the vesicles are non replicative, all dangers related to severe infections and spread of the vaccine organism are eliminated. The antigens are also presented in a natural and non-denaturated state enabling the recognition of all antigenic determinants found also on the surface of pathogenic bacteria, including conformational epitopes.

As described herein above, it is preferred that the vaccine is administered mucosally. By administering the vaccine on the mucosal surfaces (inhalation, spray etc.) the local immune response will be triggered and homing is directed to the mucosal membranes besides also stimulating systemic responses. This way, colonization resistance is much easier to achieve than by parenteral vaccination. This route of administration also allows for faster development and less pure production, as the mucosal barrier is not broken by the vaccine itself. Small amounts of contaminations will not have deletious effects such as with parenteral application.

In summary, vaccination with vesicles provides fast and efficient immune responses directed towards the sites where the infection can be expected to begin (mucosal membranes). Colonization resistance can be achieved and neutralizing antibodies against a wide variety of antigenic determinants including conformational and sugar antigens are elucidated. For human vaccination, simple administration methods such as e.g. via a nasal spray could be used, thereby additionally eliminating the risk associated with the use of needles.

The same applies also to vaccinations in veterinary medicine. However, here the focus most often is on mass immunization. This is most easy to perform by aerosolizing the particles and inhalation by lifestock. Effective local and systemic responses can thus be induced.

Employing the method of the present invention, a strain-specific vaccine can be provided quickly, e.g. in as little as a few days from isolating the bacterial strain, and less cost-intensive as compared to methods currently employed in the art, such as e.g. ultracentrifugation.

Moreover, antigens or epitopes do not have to be purified nor identified, as the presentation of the bacterial membrane vesicles is sufficient to trigger a strong and specific immune response. Using the method of the present invention, epidemics such as the EHEC epidemic in May/June 2011, which was characterised by a high frequency of serious complications, including hemolytic-uremic syndrome (HUS), may have been contained more rapidly, avoiding loss of lives and serious illnesses.

In a preferred embodiment of the method of the invention, the bacterial strain is pathogenic, preferably pathogenic for humans or lifestock, most preferably for humans. Further preferably, the bacterial strain that is to be used in step (a) is obtained by the steps of (i) identifying pathogenic bacteria in a sample obtained from the subject, and (ii) isolating the pathogenic bacteria identified in step (i).

In accordance with this embodiment, a sample obtained from a patient is analysed for the presence of bacteria. Samples such as liquor, joint aspirates, abdominal liquid, blood, eye liquid or tissues from surgeries are normally free of any bacteria and, accordingly, the presence of bacteria in such samples is an indication for an infection with a pathogenic bacterium. Samples such as for example stool or lung tissue, which normally comprise non-pathogenic bacteria present in the body, need to be analysed initially to determine the species/genus of the bacteria present in order to decide whether they comprise pathogenic bacteria. Identification of the bacterial species/genus is then achieved by methods well known in the art, such as e.g. visual identification by a skilled microbiologist, tests such as e.g. Gram-staining as well as detection of enzymes, such as e.g. catalase or coagulase, hemolysis phenotyping, cytochrome oxidase detection, as well as mass spectrometry, biochemical tests based on e.g. fermentation of different metabolites, “biochemical profiling” and derivative methods derived from the classical biochemical profiling method, such as e.g. the API™ System, as well as sequencing of genetic material or transcripts thereof (RNAs).

Once pathogenic bacteria have been identified in a sample, an isolate of the pathogen is prepared in order to avoid using mixed cultures. Isolation can be achieved by any means known in the art, such as e.g. growing the obtained bacteria on bacterial culture plates and picking a single colony, which is then further grown and optionally subjected to repeated clonal selection steps in order to ensure the presence of only one bacterial strain. In addition, the above described tests, such as e.g. visual identification by a skilled microbiologist or staining tests, may be repeated to verify the presence of only one type of bacteria. It will be appreciated, however, that the precise identity of this bacterial strain does not need to be determined, as the antigenic compounds are represented by the membrane vesicles obtained by the method of the invention, irrespective of knowledge of the bacterial strain.

In a more preferred embodiment of the method of the invention, the identification of the pathogenic bacteria in step (i) comprises a method selected from the group consisting of mass spectrometry, biochemical analysis, nucleic acid sequencing and antibiotic susceptibility testing (AST).

The term “mass spectrometry”, as used herein, is defined in accordance with the pertinent art and relates to a method of measuring the mass-to-charge ratio of charged particles. Typically, the compounds to be analysed, such as e.g. the bacteria of interest, are ionised to generate charged molecules or molecule fragments and their mass-to-charge ratios is subsequently measured. Mass spectrometry is well known in the art and has been described, e.g. in Wieser et al. MALDI-TOF MS in microbiological diagnostics-identification of microorganisms and beyond (mini review). Appl Microbiol Biotechnol. 2012 February; 93(3):965-74. Epub 2011 Dec. 25.

Biochemical analysis, in accordance with this embodiment, refers to methods using profiles of biochemical reactions catalyzed by the bacteria such as e.g. the “analytical profile index” or API™, as described e.g. in Analytical Profile Index Surhone, Tennoe, Henssonow; 2011, betascript publishing ISBN-13: 978-6136489643.

Nucleic acid sequencing is well known in the art and relates to the determination of the nucleic acid sequence of the respective bacteria using well-established methods. The sequence identified may then be compared to nucleic acid sequences of known pathogenic bacteria, thereby enabling the identification of the pathogenic bacterium under investigation.

The term “antibiotic susceptibility testing”, as used herein, is defined in accordance with the pertinent art and has been described, e.g. in Leclercq et al. EUCAST expert rules in antimicrobial susceptibility testing. Clinical Microbiology and Infection; October 2011; DOI: 10.1111/j.1469-0691.2011.03703; and published on the World Wide Web under EUCAST.org as well as under clsi.org.

Generally, antibiotic susceptibility testing (AST) is carried out using a variety of different antibiotics such as for example amikacin, ampicillin, ampicillin/sulbactam, ampicillin/clavulanic acid, aztreonam, benzylpenicillin, cefoxitin, cefalotin, cefazolin, cefepime, cefotaxime, cfotetan, cefpodoxime, ceftizoxime, ceftazidime, ceftriaxone, cefuroxim, chloramphenicol, ciprofloxacin, clindamycin, daptomycin, doripenem, ertapenem, erythromycin, gentamycin, imipenem, levofloxacin, linezolid, meropenem, minocycline, moxifloxacin, nalidixic acid, nitrofurantoin, norfloxacin, oxacillin, piperacillin, piperacillin/tazobactam, quinopristin/dalfopristin, rifampicin, streptomycin, tetracycline, tigecycline, trimethoprim/sulfamethoxazole or vancomycin. The response to these antibiotics provides a specific profile, representative of a particular pathogenic species as well as representative for the specific resistance determinants of the isolate.

Antibiotic susceptibility testing may be carried out in solution as well as on plates as described in the art, e.g. in the European committee on antimicrobial susceptibility testing guidelines (published on the World Wide Web under eucast.org) or by the clinical and laboratory standards institute (published on the World Wide Web under clsi.org).

The bacterial strain isolated for use in the method of the invention can be further treated prior to its use in the method of the invention to produce competent forms, such as e.g. bacteria that can more easily be electroporated. In addition, bacteria that can more easily be transformed by other means may also be produced. This can for example be achieved by rigorous washing of mid-log phase organisms. Further preparations can include, without being limiting, washing with buffers to enable chemical competency or mixing with helper and donor strains for transformation or mixing with phages for transduction.

In a preferred embodiment of the method of the invention, the method further comprises growing the pathogenic bacterial strain obtained from a sample in culture prior to genetically engineering said strain.

Suitable cell culture methods and media have been described herein above. Preferably, the bacteria are grown in solution. In accordance with this embodiment, the number of bacteria is increased, thereby amplifying the amount of bacteria available for use in the method of the invention.

In another preferred embodiment of the method of the invention, the method further comprises introducing an inhibitor of at least one protein of the Tol-Pal system family into the bacterial strain prior to step (c).

The term “inhibitor”, as used herein, refers to a compound that reduces or abolishes the biological function or activity of the recited protein family, by interfering with a specific target protein that is part of this protein family or by interfering with the interaction between two or more target proteins. An inhibitor may perform any one or more of the following effects in order to reduce or abolish the biological function or activity of the protein to be inhibited: (i) the transcription of the gene encoding the protein to be inhibited is lowered, i.e. the level of mRNA is lowered, (ii) the translation of the mRNA encoding the protein to be inhibited is lowered, (iii) the protein performs its biochemical function with lowered efficiency in the presence of the inhibitor, and (iv) the protein performs its cellular function with lowered efficiency in the presence of the inhibitor. The “false ligand” employed in the examples appended below falls under category (iv), i.e. it represents a soluble domain of a binding partner, thereby blocking the binding of the endogenous ligand and, consequently, inhibiting the biological function mediated by the binding of said endogenous ligand.

Compounds suitable to achieve the effect described in (i) include compounds interfering with the transcriptional machinery and/or its interaction with the promoter of said gene and/or with expression control elements remote from the promoter such as enhancers.

Compounds suitable to achieve the effect described in (ii) comprise compounds suitable to interfere with the translational machinery as well as compounds affecting the stability of the mRNA to be translated.

Compounds suitable to achieve the effect described in (iii) interfere with molecular functions of the protein to be inhibited.

Compounds suitable to achieve the effect described in (iv) include compounds which do not necessarily bind directly to the target protein, but still interfere with their activity, for example by binding to and/or inhibiting the function or expression of members of a pathway which comprises the target protein. These members may be either upstream or downstream of the protein to be inhibited within said pathway.

Such compounds include, without being limiting, small molecules, antibodies, antisense constructs and constructs for performing RNA blocking (e.g. bacterial siRNA), aptamers/spiegelmers and ribozymes. Suitable compounds further include but are not limited to, peptides such as soluble peptides, including Ig-tailed fusion peptides or the peptides recited herein below as well as members of random peptide libraries (see, e.g., Lam et al. (1991) Nature 354: 82-84; Houghten et al. (1991) Nature 354: 84-86) and combinatorial chemistry-derived molecular libraries made of D- and/or L-configuration amino acids or phosphopeptides (e.g., members of random and partially degenerate, directed phosphopeptide libraries, see, e.g., Songyang et al. (1993) Cell 72: 767-778).

A “small molecule” according to the present invention may be, for example, an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. Alternatively, the “small molecule” in accordance with the present invention may be an inorganic compound. Inorganic compounds are derived from mineral sources and include all compounds without carbon atoms (except carbon dioxide, carbon monoxide and carbonates). Preferably, the small molecule has a molecular weight of less than about 2000 amu, or less than about 1000 amu such as less than about 500 amu, and even more preferably less than about 250 amu. The size of a small molecule can be determined by methods well-known in the art, e.g., mass spectrometry. The small molecules may be designed, for example, based on the crystal structure of the target molecule, where sites presumably responsible for the biological activity, can be identified and verified in in vivo assays such as in vivo high-throughput screening (HTS) assays. Such small molecules may be particularly suitable to inhibit protein-protein-interaction by blocking specific binding sites of the target molecule.

The term “antibody” as used in accordance with the present invention comprises polyclonal and monoclonal antibodies, as well as derivatives or fragments thereof, which still retain the binding specificity. Antibody fragments or derivatives comprise, inter alia, Fab or Fab′ fragments as well as Fd, F(ab′)₂, Fv or scFv fragments; see, for example Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999. The term “antibody” also includes embodiments such as chimeric (human constant domain, non-human variable domain), single chain and humanised (human antibody with the exception of non-human CDRs) antibodies.

Various techniques for the production of antibodies are well known in the art and described, e.g. in Harlow and Lane (1988) and (1999), loc. cit. Thus, the antibodies can be produced as peptidomimetics. Further, techniques described for the production of single chain antibodies (see, inter alia, U.S. Pat. No. 4,946,778) can be adapted to produce single chain antibodies specific for the target of this invention. Also, transgenic animals or plants (see, e.g., U.S. Pat. No. 6,080,560) may be used to express (humanized) antibodies specific for the target of this invention. Most preferably, the antibody is a monoclonal antibody, such as a human or humanized antibody. For the preparation of monoclonal antibodies, any technique which provides antibodies produced by continuous cell line cultures can be used. Examples for such techniques are described, e.g. in Harlow and Lane (1988) and (1999), loc. cit. and include the hybridoma technique originally developed by Köhler and Milstein Nature 256 (1975), 495-497, the trioma technique, the human B-cell hybridoma technique (Kozbor, Immunology Today 4 (1983), 72) and the EBV-hybridoma technique to produce human monoclonal antibodies (Cole et al., Monoclonal Antibodies and Cancer Therapy, Alan R. Liss, Inc. (1985), 77-96). Surface plasmon resonance as employed in the BIAcore system can be used to increase the efficiency of phage antibodies which bind to a target protein (Schier, Human Antibodies Hybridomas 7 (1996), 97-105; Malmborg, J. Immunol. Methods 183 (1995), 7-13). It is also envisaged in the context of this invention that the term “antibody” comprises antibody constructs which may be expressed in cells, e.g. antibody constructs which may be transfected and/or transduced via, inter alia, viruses or plasmid vectors.

The term “antisense nucleic acid molecule” is known in the art and refers to a nucleic acid molecule which is complementary to a target nucleic acid, i.e. a nucleic acid encoding the target protein. An antisense nucleic acid molecule in accordance with the invention is capable of interacting with the target nucleic acid, more specifically it is capable of hybridising with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901).

The term “bacterial siRNA”, as used herein, relates to sequences of non protein coding RNA molecules which act by binding to single stranded mRNA of the bacterium in order to block or interfere with their further transcription or translation or the assembly of the ribosomal complex with mRNA. It is understood that such siRNA is used to intentionally alter the expression or translation of specific complementary mRNA molecules.

Aptamers are nucleic acid or peptide molecules that bind a specific target molecule. More specifically, aptamers can be classified as nucleic acid aptamers, such as DNA or RNA aptamers, or peptide aptamers. Whereas the former normally consist of (usually short) strands of oligonucleotides, the latter preferably consist of a short variable peptide domain, attached at both ends to a protein scaffold. Whereas nucleic acid aptamers are nucleic acid molecules that are in the natural D-conformation, the corresponding nucleic acid molecules that are in the L-conformation are referred to in the art and herein as spiegelmers.

Aptamers, as well as spiegelmers, are usually created by selecting them from a large random sequence pool, but natural aptamers also exist in riboswitches. Aptamers and spiegelmers can be used as macromolecular drugs. They can be combined with ribozymes to self-cleave in the presence of their target molecule. These compound molecules have additional research, industrial and clinical applications (Osborne et. al. (1997), Current Opinion in Chemical Biology, 1:5-9; Stull & Szoka (1995), Pharmaceutical Research, 12, 4:465-483).

A ribozyme (from ribonucleic acid enzyme, also called RNA enzyme or catalytic RNA) is an RNA molecule that catalyses a chemical reaction. Many natural ribozymes catalyse either their own cleavage or the cleavage of other RNAs, but they have also been found to catalyse the aminotransferase activity of the ribosome. Non-limiting examples of well-characterised small self-cleaving RNAs are the hammerhead, hairpin, hepatitis delta virus, and in vitro-selected lead-dependent ribozymes, whereas the group I intron is an example for larger ribozymes. The principle of catalytic self-cleavage has become well established in the last decade. The hammerhead ribozymes are characterised best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: An interesting region of the RNA, which contains the GUC (or CUC) triplet, is selected. Two oligonucleotide strands, each usually with 6 to 8 nucleotides, are taken and the catalytic hammerhead sequence is inserted between them. Molecules of this type were synthesised for numerous target sequences. They showed catalytic activity in vitro and in some cases also in vivo. The best results are usually obtained with short ribozymes and target sequences.

Also useful in accordance with the present invention, is the combination of an aptamer recognizing a small compound with a hammerhead ribozyme. The conformational change induced in the aptamer upon binding the target molecule is supposed to regulate the catalytic function of the ribozyme.

The term “peptide”, as used herein, describes a group of molecules consisting of up to 30 amino acids, whereas the term “protein”, as used herein, describes a group of molecules consisting of more than 30 amino acids. Peptides and proteins may further form dimers, trimers and higher oligomers, i.e. consisting of more than one molecule which may be identical or non-identical. The corresponding higher order structures are, consequently, termed homo- or heterodimers, homo- or heterotrimers etc. The terms “peptide” and “protein” (wherein “protein” is interchangeably used with “polypeptide”) also refer to naturally modified peptides/proteins wherein the modification is effected e.g. by glycosylation, acetylation, phosphorylation and the like. Such modifications are well-known in the art.

Also encompassed herein are modified versions of these inhibitory compounds.

The term “modified versions of these inhibitory compounds” in accordance with the present invention refers to versions of the compounds that are modified to achieve i) modified spectrum of activity, organ specificity, and/or ii) improved potency, and/or iii) decreased toxicity (improved therapeutic index), and/or iv) decreased side effects, and/or v) modified onset of therapeutic action, duration of effect, and/or vi) modified pharmacokinetic parameters (resorption, distribution, metabolism and excretion), and/or vii) modified physico-chemical parameters (solubility, hygroscopicity, color, taste, odor, stability, state), and/or viii) improved general specificity, organ/tissue specificity, and/or ix) optimised application form and route by, for example, (a) esterification of carboxyl groups, or (b) esterification of hydroxyl groups with carboxylic acids, or (c) esterification of hydroxyl groups to, e.g. phosphates, pyrophosphates or sulfates or hemi-succinates, or (d) formation of pharmaceutically acceptable salts, or (e) formation of pharmaceutically acceptable complexes, or (f) synthesis of pharmacologically active polymers, or (g) introduction of hydrophilic moieties, or (h) introduction/exchange of substituents on aromates or side chains, change of substituent pattern, or (i) modification by introduction of isosteric or bioisosteric moieties, or (j) synthesis of homologous compounds, or (k) introduction of branched side chains, or (k) conversion of alkyl substituents to cyclic analogues, or (l) derivatisation of hydroxyl groups to ketales, acetales, or (m) N-acetylation to amides, phenylcarbamates, or (n) synthesis of Mannich bases, imines, or (o) transformation of ketones or aldehydes to Schiff's bases, oximes, acetales, ketales, enolesters, oxazolidines, thiazolidines; or combinations thereof.

The various steps recited above are generally known in the art. They include or rely on quantitative structure-action relationship (QSAR) analyses (Kubinyi, “Hausch-Analysis and Related Approaches”, VCH Verlag, Weinheim, 1992), combinatorial biochemistry, classical chemistry and others (see, for example, Holzgrabe and Bechtold, Deutsche Apotheker Zeitung 140(8), 813-823, 2000).

Preferably, the activity of the respective target protein of the Tol-Pal system family is inhibited such that it has less than 90%, more preferred less than 80%, less than 70%, less than 60% or less than 50% of the activity as compared to the activity it has in the absence of any inhibition. Even more preferred is that its activity is reduced such that it is less than 25%, more preferred less than 10%, less than 5%, or less than 1% of the activity as compared to the activity it has in the absence of any inhibition. Most preferably, the activity of the target protein is fully inhibited, i.e. no expression or activity is detectable.

The efficiency of an inhibitor can be quantified by comparing the level of expression and/or activity in the presence of an inhibitor to that in the absence of the inhibitor. For example, as a measure may be used: the change in amount of mRNA formed, the change in amount of protein formed, the change in biological activity of the target proteins as described herein below, and/or the change in the cellular phenotype or in the phenotype of an organism. In other words, the efficiency of an inhibitor can be quantified by comparing e.g. the amount of target protein in the presence of an inhibitor to that in the absence of the inhibitor or by determining the biological activity of the target protein present prior to and after administration of the inhibitor, wherein a reduction in the amount or biological activity of the target protein in the presence of or after administration of the inhibitor as compared to in the absence of or prior to said administration is indicative of a successful inhibition of the target protein. Means and methods to determine the amount of mRNA or proteins in a sample or for determining biological activities are well known in the art.

Methods for determining the expression of a protein on the nucleic acid level include, but are not limited to, northern blotting, PCR, RT-PCR or real RT-PCR. Methods for the determination of the expression of a protein on the amino acid level include but are not limited to western blotting or polyacrylamide gel electrophoresis in conjunction with protein staining techniques such as Coomassie Brilliant blue or silver-staining. Also of use in protein quantification is the Agilent Bioanalyzer technique. These methods are well known in the art.

The determination of binding of potential inhibitors can be effected in, for example, any binding assay, preferably biophysical binding assay, which may be used to identify binding of test molecules prior to performing the functional/activity assay with the inhibitor. Suitable biophysical binding assays are known in the art and comprise fluorescence polarisation (FP) assay, fluorescence resonance energy transfer (FRET) assay and surface plasmon resonance (SPR) assay. For example, a modulator acting via binding to an enzyme, and thereby modulating the activity of said enzyme, may be tested by FRET by labelling either the modulator or the enzyme with a donor chromophore and the other molecule with an acceptor chromophore. These chromophore-labelled molecules are then mixed with each other. When they are dissociated, donor emission can be detected upon donor excitation at the appropriate wavelength. However, when the donor and acceptor are in proximity (1-10 nm) due to the interaction of the modulator with the enzyme, the acceptor emission is predominantly observed because of the intermolecular FRET from the donor to the acceptor.

The function of an inhibitor suitable for the method of the present invention may be identified and/or verified by using high throughput screening assays (HTS). High-throughput assays, independently of being biochemical, cellular or other assays, generally may be performed in wells of microtiter plates, wherein each plate may contain, for example 96, 384 or 1536 wells. Handling of the plates, including incubation at temperatures other than ambient temperature, and bringing into contact of test compounds with the assay mixture is preferably effected by one or more computer-controlled robotic systems including pipetting devices. Where large libraries of test compounds are to be screened and/or screening is to be effected within short time, mixtures of, for example 10, 20, 30, 40, 50 or 100 test compounds may be added to each well. In case a well exhibits biological activity, said mixture of test compounds may be de-convoluted to identify the one or more test compounds in said mixture giving rise to the observed biological activity.

In accordance with the present invention, the inhibitory molecules may be introduced into the bacteria by any suitable method known in the art, such as e.g. transformation, transfection, active or passive transport through the membrane and other methods described elsewhere herein.

Most preferably, introduction is via transformation of the bacterial strain with a nucleic acid molecule encoding the inhibitor or a nucleic acid molecule that is an inhibitor per se, such as e.g. constructs involved in RNA interference and antisense nucleic acid molecules. It will be appreciated that where a nucleic acid molecule encoding the inhibitor is employed, expression of said inhibitor from the nucleic acid molecule has to be ensured. Means and methods of ensuring expression of a nucleic acid molecule have been described herein above.

As used herein, the term “Tol-Pal system family”, also referred to herein as the “Tol-Pal system”, refers to a system comprised of several proteins, including TolQ, TolR, Pal, TolB, TolA, and may additionally include ybgC, ybgF or amiA. There are two inner membrane transmembrane proteins involved, TolQ and TolR. TolR is known to dimerize and bind to TolQ. This binding is needed to energize TolA which is completely located in the periplasm and spans trough the peptidoglycan layer. Interaction between TolR and TolQ takes place via a periplasmic loop. The outer membrane associated Pal protein interacts with TolA and TolB, while TolA also interacts directly with TolB. Pal may have an additional interaction with other outer membrane proteins such as OmpA. Some bacterial groups also harbour one or more of the genes ybgC, ybgF or amiA. The core tol-pal cluster is present in almost all gram-negative bacteria, except Neisseria. All genes have homologues which are sometimes only identified by their position in the cluster. The Tol Pal system has been described in the art, e.g. in Cascales E, Lloubès R.; Deletion analyses of the peptidoglycan-associated lipoprotein Pal reveals three independent binding sequences including a TolA box. Mol. Microbiol. 2004 February; 51(3):873-85; Cascales E, Lloubès R, Sturgis J N.; The TolQ-TolR proteins energize TolA and share homologies with the flagellar motor proteins MotA-MotB. Mol. Microbiol. 2001 November; 42(3):795-807; Journet L, Rigal A, Lazdunski C, Bénédetti H. Role of TolR N-terminal, central, and C-terminal domains in dimerization and interaction with TolA and tolQ. J. Bacteriol. 1999 August; 181(15):4476-84; Zhang X Y, Goemaere E L, Seddiki N, Célia H, Gavioli M, Cascales E, Lloubes R.; Mapping the interactions between Escherichia coli TolQ transmembrane segments. J Biol. Chem. 2011 Apr. 1; 286(13):11756-64. Epub 2011 Feb. 1; James N. Sturgis: organisation and evolution of tol-pal gene cluster J. Mol. Microbiol. Biotechnol. 2001 3(1) 113-122).

Accordingly, the term “inhibitor of at least one protein of the Tol-Pal system family” refers to an inhibitor of any one of the above recited molecules that form part of this family.

It will be appreciated that the additional step of introducing an inhibitor of at least one protein of the Tol-Pal system family into the bacterial strain prior to step (c) may be carried out either concomitantly with the genetical engineering with the nucleic acid molecule encoding a fusion protein, or at a different time, i.e. prior to or after the genetical engineering with the nucleic acid molecule encoding a fusion protein. Accordingly, the additional step of introducing an inhibitor of at least one protein of the Tol-Pal system family into the bacterial strain may be carried out prior to the genetical engineering of step (a), with the genetic engineering in step (a) or after the genetic engineering of step (a), i.e. either before or after the growth culture of step (b). Preferably, step (a) encompasses both the introduction of the inhibitor and of the nucleic acid molecule encoding the fusion protein, either simultaneously or subsequently to each other. Most preferably, the inhibitor and the nucleic acid molecule encoding the fusion protein are introduced simultaneously.

Due to the inhibition of at least one protein of the Tol-Pal system family, the production of OMVs from the individual bacteria is enhanced, thereby facilitating enrichment and providing a purer and increased amount of vesicles for formulation into a vaccine.

Established methods in the art have focused on mutations in known target genes, in order to destabilise the connection between the outer and inner membrane (Henry et al. 2004). However, this process is time consuming and not for every genus there are established protocols to perform chromosomal mutations. Moreover, the method described by Henry et al. 2004 emphasises the importance of growing bacteria on plates, which results in insufficient amounts of bacteria.

Thus, in accordance with the present invention, an inhibitor is employed in order to destabilise the connection between the outer and inner membrane of Gram-negative bacteria, thereby enhancing the formation of OMVs. In accordance with the present invention, it is preferred to induce the expression of proteins that act as “false ligands”, i.e. which disrupt at least partially the membrane integrity systems. Insertion of such factors can be achieved with plasmids which can be transformed effectively.

To insert false ligands into the periplasm, sec or tat leader sequences can be used, as described herein above. In the appended examples, the use of a derivate of TorA, i.e. a part of the TolR protein, as the false ligand is shown. The sequence-stretch for TolR can be individually cloned for each species. A synthetic multi-blocking protein comprising false ligand sequences of different species separated by spacers to be used unchanged in a broad range of species may also be employed.

In a more preferred embodiment of the method of the invention, the inhibitor of at least one protein of the Tol-Pal system family is a small molecule inhibitor, a ribozyme, an RNAi agent, an antisense construct, an antibody, a spiegelmer or an aptamer.

In an alternative more preferred embodiment of the method of the invention, the inhibitor of at least one protein of the Tol-Pal system family is selected from the group consisting of a soluble TolA and/or TolR periplasmic domain, the N-terminal domain of a group A colicin and the minor coat protein g3p.

The term “soluble TolA periplasmic domain” is defined in accordance with the pertinent art and relates to the part of TolA which is located in the periplasmic space. This part of TolA is the amino acid sequence of TolA starting after the C-terminal end of the N-terminal transmembrane segment. The soluble TolA periplasmic domain has been described in the art (Cascales 2001, Molecular Microbiology 42(3), 795-807; Walbuger 2002, Molecular Microbiology 44(3), 695-708) and has an amino acid sequence as shown in SEQ ID NOs:31 to 53. Full length TolA amino acid sequence sequences are shown in SEQ ID NOs: 7 to 30.

The inhibitory effect of a soluble TolA periplasmic domain is mediated by binding to other partners of the Tol-Pal system such as TolB, Pal, TolR or TolQ. The exact interaction points for each individual species has not yet been characterized. However, false ligands for all those interactions will cause disruption of the system and a hypervesiculation phenotype.

The term “soluble TolR periplasmic domain” is defined in accordance with the pertinent art and relates to the periplasmic loop of TolR which is located at the C-terminal end of the N-terminal transmembrane domain of TolR; represented by residue 44-117 of the protein described in AAN79290 in E. coli.

The soluble TolR periplasmic domain has been described in the art in (Cascales 2001; Sturgis et al. 2001 J. Microbiol. Biotechnol. 3(1), 113-122) and has an amino acid sequence as shown in SEQ ID NOs: 77 to 99. Full length TolR amino acid sequence sequences are shown in SEQ ID NOs: 54 to 76.

The soluble TolR periplasmic domain inhibits the interaction between TolR and TolA, which leads to a loss of energy transfer into the Tol Pal system, thus blocking its function.

The term “group A colicin” is defined in accordance with the pertinent art and relates to a group of proteins produced by bacteria to kill other bacteria by binding to their outer membrane proteins, entering and depolarizing their membranes. A prototypic group A colicin has been described in the art, e.g. by Morlon et al. Complete Nucleotide sequence of the Structural Gene for Colicin A, a Gene Translated at Non-uniform Rate; J. Mol. Biol. (1983) 170, 271-285 and has the amino acid sequence and the nucleic acid sequence as described in detail in Chartier et al, J. Mol. Biol. (1983) 170, 271-285.

The N-terminal domain of a group A colicin refers to the amino acid residues 29 to 107 of the Colicin A as described in Chartier et al, J. Mol. Biol. (1983) 170, 271-285 and is shown in SEQ ID NOs: 100 and 101.

The N-terminal domain of a group A colicin binds to the periplasmic C-terminal domain of TolA as well as to TolB and partly to TolR. These interactions interfere with the normal function of the Tol-Pal system and, thereby, enhance the outer membrane vesicle production.

The term “minor coat protein g3p” is defined in accordance with the pertinent art and relates to the N-terminal domain of M13 phage g3p protein. This part of g3p binds to the D3 domain of TolA. The minor coat protein g3p has been described in the art as part of the entry mechanism of M13 filamentous phage in e.g. Lubkowski J et al. 1999, Structure 7:711-722; Filamentous phage infection: crystal structure of g3p in complex with its coreceptor, the C-terminal domain of TolA. It has the amino acid sequence as shown in SEQ ID NO: 102 and the nucleic acid sequence as shown in SEQ ID NO: 103; as also shown in the GenBank database entry: AB591081.1; publication date Aug. 2, 2011.

The minor coat protein g3p binds with its N1 domain to the D3 domain of TolA and triggers local changes in membrane integrity leading to the uptake of bacteriophage M13 during natural infection. This mechanism presumably interferes with other interactions of the TolA periplasmic domain leading not only to the entrance of the phage DNA but also to enhanced production of outer membrane vesicles.

In a further more preferred embodiment of the method of the invention, the at least one protein of the Tol-Pal system is selected from the group consisting of TolA, TolB, TolQ, TolR, Pal, Lpp, ybgC and NlpI.

“TolA”, as used throughout the present invention, is defined in accordance with the pertinent art and relates to the protein described in E. coli as well as its homologues in other species. TolA is represented in E. coli by the GenBank accession number AAA24683 (publication date Apr. 26, 1993) and has been described in the art, e.g. in Cascales 2001, Molecular Microbiology 42(3), 795-807; Walbuger 2002, Molecular Microbiology 44(3), 695-708.

In accordance with the present invention, “TolB”, is defined in accordance with the pertinent art and relates to the protein as it is described in E. coli and its homologues in other species. TolB is represented in E. coli by the GenBank accession number AAA24684 (publication date Apr. 26, 1993) and has been described in the art, e.g. in James N. Sturgis: organisation and evolution of tol-pal gene cluster J. Mol. Microbiol. Biotechnol. 2001 3(1) 113-122.

“TolQ”, as used throughout the present invention, is defined in accordance with the pertinent art and relates to the protein as it is described in E. coli and its homologues in other species. TolQ is represented in E. coli by the GenBank accession number AAN79289 (publication date Nov. 21, 2011) and has been described in the art, e.g. in James N. Sturgis: organisation and evolution of tol-pal gene cluster J. Mol. Microbiol. Biotechnol. 2001 3(1) 113-122.

“TolR”, as used throughout the present invention, is defined in accordance with the pertinent art and relates to the protein as it is described in E. coli and its homologues in other species. TolR is represented in E. coli by the GenBank accession number AAC73832 (publication date Sep. 1, 2011) and has been described in the art, e.g. in James N. Sturgis: organisation and evolution of tol-pal gene cluster J. Mol. Microbiol. Biotechnol. 2001 3(1) 113-122.

In accordance with the present invention, “Pal”, is defined in accordance with the pertinent art and relates to the protein as it is described in E. coli and its homologues in other species. Pal is represented in E. coli by the NCBI Reference Sequence YP_{—}851845 (publication date Jan. 26, 2012) and has been described in the art, e.g. in James N. Sturgis: organisation and evolution of tol-pal gene cluster J. Mol. Microbiol. Biotechnol. 2001 3(1) 113-122.

In accordance with the present invention, “Lpp”, is defined in accordance with the pertinent art and relates to the protein as it is described in E. coli and its homologues in other species. Lpp is represented in E. coli by the NCBI Reference Sequence YP_{—}001462971 (publication date Jan. 24, 2012) and has been described in the art, e.g. in Rasko et al. The pangenome structure of Escherichia coli: Comparative Genomic Analysis of E. coli Commensal and Pathogenic Isolates; J. Bacteriol. 2008, 190 (20):6881.DOI:10.1128/JB.00619-08.

In accordance with the present invention, “ybgC”, is defined in accordance with the pertinent art and relates to the protein as it is described in E. coli and its homologues in other species. ybgC is represented in E. coli by the GenBank accession number AAN79288 (publication date Nov. 21, 2011) and has been described in the art, e.g. in Welch et al. Extensive mosaic structure revealed by the complete genome sequence of uropathogenic Escherichia coli. PNAS, 2002 24; 99(26):17020-4.

“NlpI”, as used throughout the present invention, is defined in accordance with the pertinent art and relates to the protein as it is described in E. coli and its homologues in other species. NlpI is represented in E. coli by the GenBank accession number ABV07583 (publication date Feb. 13, 2011) and has been described in the art, e.g. in Rasko et al. The pangenome structure of Escherichia coli: Comparative Genomic Analysis of E. coli Commensal and Pathogenic Isolates; J. Bacteriol. 2008, 190 (20):6881.DOI:10.1128/JB.00619-08.

In another preferred embodiment of the method of the invention, the genetic engineering further comprises the introduction of additional immunogenic determinants for expression in or on the membrane vesicles obtained from the bacteria.

The term “immunogenic determinants”, as used herein, relates to protein-antigens as well as lipids, phospholipids, lipopolysaccharides and sugar moieties as well as additional capsule molecules. Whereas immunogenic proteins can be encoded directly by respective gene sequences, it will be understood that lipids, phospholipids, lipopolysaccharides and sugar moieties or capsule molecules can be inserted by inserting gene sequences coding for proteins (enzymes) catalysing their production from simple organic or anorganic molecules, or by modification from existing precursor forms within the cell.

In accordance with this embodiment, either (i) the nucleic acid molecule encoding the fusion protein may comprise further sequences that are presented on the outside of the membrane vesicles or (ii) an additional nucleic acid molecule is employed encoding a fusion protein comprising a bacterial membrane protein fused to an immunogenic determinant of interest. Also encompassed by this embodiment is the use of nucleic acid molecules encoding the immunogenic determinant of interest and, optionally, a leader sequence ensuring the transport of the translated expression product into e.g. the periplasm of the bacteria in order to ensure enclosure within or presentation on the membrane vesicles.

All of the definitions and preferred embodiments recited herein above apply mutatis mutandis also to this preferred embodiment of the method of the invention. Means and methods of altering nucleic acid molecules and of ensuring expression and targeting in the correct cellular domain have also been described elsewhere herein.

In another preferred embodiment of the method of the invention, the pathogenic bacterial strain is a Gram-negative or a Gram-positive bacterial strain.

The characterisation of bacteria based on Gram staining is well known in the art. Gram-positive bacteria are those where the crystal violet staining is fixed/complexed firmly in the cell wall's peptidoglycan structure by Gram's iodine solution so that it cannot be removed by washing with acetone or ethanole. They therefore remain dark blue whereas the gram-negative bacteria can be destained and will appear red by the counter staining with safranin and fuchsine. The difference in Gram staining is based on the structural differences between Gram-positive and Gram-negative bacteria. In other words, Gram-positive bacteria typically lack the outer membrane found in Gram-negative bacteria and the Gram staining is positive because of the high amount of peptidoglycans in the cell wall.

Gram-positive bacteria are generally characterised by having a cytoplasmic lipid membrane and a wide peptidoglycan layer, wherein the individual peptidoglycan molecules are cross-linked by pentaglycine chains by a DD-transpeptidase enzyme. In some species, capsule polysaccharides are present and some species have a flagellum (Madigan, M. and Martinko, J. (editors). (2005). Brock Biology of Microorganisms (11th ed.). Prentice Hall. ISBN 0131443291).

Non-limiting examples of Gram-positive bacteria include the Actinobacteria, such as e.g. Corynebacterium, Mycobacterium, Nocardia and Streptomyces as well as the Firmicutes, such as for example Staphylococcus, Streptococcus, Enterococcus, Bacillus, Clostridium and Listeria.

Gram-negative bacteria are generally characterised by having a cytoplasmic membrane, a thinner peptidoglycan layer than Gram-positive bacteria and an outer membrane outside the peptidoglycan layer containing lipopolysaccharide (LPS) containing porins, that act as pores for certain molecules. Between the peptidglycan layer and the outer cell membrane is the so-called periplasmic space. Furthermore, teichoic acids or lipoteichoic acids, which are present in Gram-positive bacteria, are absent in Gram-negative bacteria.

Non-limiting examples of Gram-negative bacteria include Escherichia, Pseudomonas, Klebsiella, Stenotophomonas, Salmonella, Neisseria, Hemophilus, Shigella, Yersinia, Moraxella, Helicobacter, Stenotrophomonas, Bdellovibrio, acetic acid bacteria, Proteus, Enterobacter, Serratia, Helicobacter, Legionella, Edwardsiella and Acinetobacter. Additional groups of Gram-negative bacteria include, without being limiting, spirochaetes, green sulfur and green non-sulfur bacteria.

In a more preferred embodiment of the method of the invention, the Gram-positive bacterial strain is selected from the group consisting of Staphylococcus, Streptococcus, Enterococcus, Bacillus and Clostridium.

Preferred species of the above recited genera are Staphylococcus aureus MRSA, Streptococcus pneumoniae, Enterococcus faecium VRE, Bacillus anthracis or Clostridium difficile.

In another more preferred embodiment of the method of the invention, the Gram-negative bacterial strain is selected from the group consisting of Escherichia, Enterobacter, Klebsiella, Pseudomonas, Acinetobacter, Stenotophomonas, Salmonella, Shigella and Yersinia.

Preferred species of the above recited genera are E. coli, Enterobacter cloacae, Shigella flexneri, Shigella sonnei, Shigella boydii, Shigella dysenteriae, P. aeruginosa group, P. putida group, P. fluorescens group, P. stutzeri group, K. pneumoniae, K. oxytoca, S. enterica of all different serovars such as e.g. Salmonella typhimurium; Salmonella typhi, Salmonella paratyphi, Y. pestis, Y. enterocolitica, Y. pseudotuberculosis, A. calcoaceticus-baumanii group, A. lwoffi, A. haemolyticus, S. Stenotrophomonas maltophilia and Francisella tularensis.

Preferably, the bacteria are Gram-negative bacteria. Even more preferably, the bacteria are selected from the group consisting of the genera Escherichia, Klebsiella, Acinetobacter and Pseudomonas. Most preferably, the bacteria are selected from the group consisting of E. coli, P. aeruginosa, P. putida, K. pneumoniae, K. oxytoca and A. baumanii.

In another preferred embodiment of the method of the invention, the bacterial membrane protein is selected from the group consisting of membrane pore-forming proteins, auto-transporter proteins, receptor proteins and a protein comprising or consisting of the sequence of SEQ ID NO:1 or a functional variant thereof.

The term “membrane pore-forming protein”, as used herein, is defined in accordance with the pertinent art and relates to proteins forming channels (pores) through membranes of bacteria by inserting into the membrane and opening a channel. Non-limiting examples of membrane pore-forming proteins include ClyA, staphyloccus alphaHL, LukF or LLO. Membrane pore-forming proteins have been described in the art, e.g. in Gilbert, R. J. (2002). “Pore-forming toxins.” Cell Mol Life Sci 59(5): 832-44. b and Delcour, A. H. (2002). “Structure and function of pore-forming beta-barrels from bacteria.” J Mol Microbiol Biotechnol 4(1): 1-10.

The term “auto-transporter protein”, as used herein, is defined in accordance with the pertinent art and relates to proteins which are capable of translocating the N-terminal passenger domain through the membrane by means of their own C-terminal sequence (translocator domain). The N-terminal passenger domain can be cleaved from the natural translocator domain and/or can be associated with the translocator domain. Auto-transporter proteins have been described in the art, e.g. in Henderson et al. 1998 The great escape: structure and function of the autotransporter proteins, Trends in Microbiol 6 (9): 370-378.

Non-limiting examples of auto-transporter proteins (and their database accession numbers) include Ssp (P09489) Ssp-h1 (BAA33455), Ssp-h2 (BAA11383), PspA (BAA36466), PspB (BAA36467), Ssa1 (AAA80490), SphB1 (CAC44081), AspA/NalP (AAN71715), VacA (Q48247), AIDA-I (Q03155), IcsA (AAA26547), MisL (AAD16954), TibA (AAD41751), Ag43 (P39180), ShdA (AAD25110), AutA (CAB89117), Tsh (154632), SepA (CAC05786), EspC (AAC44731), EspP (CAA66144), Pet (AAC26634), Pic (AAD23953), SigA (AAF67320), Sat (AAG30168), Vat (AA021903), EpeA (AAL18821), EatA (AA017297), EspI (CAC39286), EaaA (AAF63237), EaaC (AAF63038), Pertactin (P14283), BrkA (AAA51646), Tef (AAQ82668), Vag8 (AAC31247), PmpD (084818), Pmp20 (Q9Z812), Pmp21 (Q9Z6U5), IgA1 Neisseria protease (NP_{—}283693), App (CAC14670), IgA1 Haemophilus protease (P45386), Hap (P45387), rOmpA (P15921), rOmpB (Q53047), ApeE (AAC38796), EstA (AAB61674), Lip-1 (P40601), McaP (AAP97134), BabA (AAC38081), SabA (AAD06240), AlpA (CAB05386), Aae (AAP21063) or NanB (AAG35309), as reviewed in Henderson et al. 2004 Microbiology and molecular biology reviews p. 692-744, Type V protein secretion pathway: the autotransporter story.

The term “receptor protein”, as used herein, is defined in accordance with the pertinent art and relates to proteins protruding towards the outside of the outermost bacterial membrane to enable binding of substances (proteins or small molecules) from outside the cell.

Non-limiting examples of receptor proteins include the siderophore receptors, OmpA-like transmembrane domain containing proteins, virulence-related outer membrane proteins of the OmpX family (see e.g. Mecsas et al. Identification and characterization of an outer membrane protein, OmpX, in Escherichia coli that is homologous to a family of outer membrane proteins including Ail of Yersinia enterocolitica. J. Bacteriol. (1995)), of the outer membrane protein W family (OmpW; see e.g. Pilsl et al. Characterization of colicin S4 and its receptor, OmpW, a minor protein of the Escherichia coli outer membrane. J. Bacteriol. 1999 June; 181(11):3578-81 PMID: 10348872) as well as members of the antimicrobial peptide resistance and lipid A acylation protein family (PagP; see e.g. Hwang et al. Solution structure and dynamics of the outer membrane enzyme PagP by NMR. Proc Natl Acad Sci USA 2002; 99:13560-13565) and Opacity family porins (NspA; see e.g. Vandeputte-Rutten et al.; Crystal structure of Neisserial surface protein A (NspA), a conserved outer membrane protein with vaccine potential. J Biol. Chem. 2003 Jul. 4; 278(27):24825-30. Epub 2003 Apr. 26). Alternatively, and in accordance with this embodiment, the bacterial membrane protein comprises or consists of the sequence of SEQ ID NO:1 or a functional variant thereof.

In those embodiments where the bacterial membrane protein comprises (rather than consists of) the sequence of SEQ ID NO:1 or a functional variant thereof, additional amino acids extend over the specific sequence either at the N-terminal end or the C-terminal end or both. Preferably, no more than 500 additional amino acids are present at the N-terminal end and no more than 500 additional amino acids are present at the C-terminal end. More preferably no more than 400, such as no more than 300, more preferably no more than 200, such as no more than 100, no more than 90, no more than 80, no more than 70, no more than 60, no more than 50, no more than 40, no more than 30, no more than 20 and even more preferably no more than 10 additional amino acids are independently present at either one or both of the N- or C-terminal end. Most preferably, no more than 5 additional amino acids are independently present at either one or both of the N- or C-terminal end.

Where such additional amino acids are comprised in the membrane protein, it is preferred that the membrane protein having these additional amino acids maintains or essentially maintains the biological function of the membrane protein consisting of the sequence of SEQ ID NO:1.

The requirement that the membrane protein having these additional amino acids “essentially maintains the biological function of the membrane protein” refers to the requirement that the resulting membrane protein maintains at least 50%, such as at least 60%, more preferably at least 70%, such as at least 80%, such as for example at least 85%, such as at least 90%, such as at least 95%, and more preferably at least 98% of the biological function of the membrane protein consisting of the sequence of SEQ ID NO:1. Most preferably, the biological function of the resulting membrane protein is fully maintained, i.e. to 100%, or is improved, i.e. showing more than 100% of the biological function of the membrane protein consisting of the sequence of SEQ ID NO:1.

The term “biological function”, as used herein in relation to a membrane protein, relates to the capability of said protein to integrate into, or associate with a membrane. The term “membrane”, as used herein, relates to any membrane naturally occurring in bacteria or bacterial vesicles, such as the above described membrane vesicles as well as to artificial membrane systems, including, without being limiting, liposomes, artificial bilayers of phospholipids, isolated plasma membrane such as cell membrane fragments or cell membrane fractions. Preferably, the membrane is a membrane vesicle. The efficiency of integration into a membrane or association therewith can be measured by methods well known in the art. For example, a labelled candidate membrane protein can be incubated with one side of a bilayer or in a suspension of liposomes and the accumulation of membrane protein with time can be measured, using appropriate means to detect the label (e.g., scintillation counting of medium on each side of the bilayer, or of the contents of liposomes isolated from the surrounding medium). Alternatively, bacteria may be transformed with a vector encoding the candidate membrane protein and the integration into or association with the membrane of membrane vesicles derived from said bacteria can be determined, for example by employing a labelling approach as described above or by staining the membrane vesicles with e.g. an appropriate antibody for the respective membrane protein and performing electron microscopy or fluorescence microscopy of membrane vesicle isolations labelled with appropriate antibodies. Further methods include e.g. the creation of fusions of a membrane protein with fluorescence molecules or attachment sites of those, such as e.g. a tag for FlaSH.

A direct comparison of the biological function can be achieved by carrying out the same experiment using a membrane protein consisting of the amino acid sequence of SEQ ID NO:1. However, it will be appreciated that standard values for a membrane protein consisting of the amino acid sequence of SEQ ID NO:1 may generated in advance and stored for comparison, thereby rendering the parallel comparison unnecessary if the same experimental conditions are observed.

The membrane protein consisting of the amino acid sequence of SEQ ID NO:1 corresponds to the membrane protein employed in the examples described herein below. Preferably, said membrane protein is encoded by the nucleic acid sequence shown in SEQ ID NO:2. However, it will be appreciated that further nucleic acid sequences are also suitable to encode the amino acid sequence of SEQ ID NO:1. This is due to the degeneracy of the genetic code and the fact that certain bacteria may show an improved translation of the desired membrane protein if the encoding nucleic acid sequence is codon optimised for the translational machinery of these particular bacteria. All these nucleic acid sequences are also encompassed herein for use in generating the membrane protein in accordance with the present invention or fusion proteins comprising said membrane protein.

The membrane protein consisting of SEQ ID NO:1 is a novel membrane protein designed by the inventors of the present invention. This protein provides the advantage that it is not specific for one particular bacterial species or strain, but is universally employable in a wide range of organisms, such as e.g. E. coli, Yersinia enterocolitica, Klebsiella pneumoniae, Salmonella enteritidis, Pseudomonas aeruginosa.

The term “functional variant”, as used herein, relates to a membrane protein differing in its amino acid sequence from the sequence of SEQ ID NO:1 but maintaining or essentially maintaining the biological activity of the membrane protein consisting of SEQ ID NO:1. Means and methods to determine whether a membrane protein maintains this activity have been described herein above. The definition with regard to the term “essentially maintaining the biological activity” is as defined herein above. Preferably, the amino acid sequence of the functional variant has at least 85% sequence identity to the amino acid sequence of SEQ ID NO:1, more preferably at least 90%, such as for example at least 95%, mores preferably 97%, even more preferably at least 98% and most preferably at least 99% sequence identity to the amino acid sequence of SEQ ID NO:1.

Preferably, the bacterial membrane protein in accordance with the present invention is a protein comprising or consisting of the sequence of SEQ ID NO:1. Even more preferably, the bacterial membrane protein is a protein consisting of the sequence of SEQ ID NO:1.

In another preferred embodiment of the method of the invention, the at least one affinity tag is selected from the group consisting of a Strep-tag, chitin binding protein (CBP), maltose binding protein (MBP), glutathione-S-transferase (GST), FLAG-tag, HA-tag, Myc-tag, His-tag and derivatives thereof.

The skilled person knows how to employ any of these tags and all these tags are well known and have been described herein above.

Derivatives of the specifically recited tags include, without being limiting the strep ONE tag as described on the World Wide Web site iba-go.com and other tags comprising several similar tag sequences in close proximity or more than one different tag sequence in close proximity.

Preferably, the affinity tag employed in the method of the present invention is the Strep ONE tag, which is sold e.g. by IBA BioTAGnology and has the amino acid sequence SAWSHPQFEK(GGGS)₂GGSAWSHPQFEK (SEQ ID NO: 103).

This tag has been approved for human medicine and is therefore particularly suitable for human use. In addition, the Strep ONE tag allows the elution of the bound particles without the need for denaturing conditions, therefore enabling the purification of native vesicles.

It will be appreciated that where different tags are used that are not approved for use in human, or veterinary medicine, such tags can be removed prior to administration by cleavage, e.g. enzymatic cleavage, of the interaction domain inside the purification system. This can e.g. be achieved by inserting a cleavage domain sequence inbetween the tag and the region linking the tag to the bacterial membrane protein. Enzymatic cleavage can then be used to remove the tag after binding between the tag and the purification matrix has occurred, thereby eluting the vesicle fraction without the tag sequence.

In another preferred embodiment of the method of the invention, the fusion protein comprises or consists of the amino acid sequence of SEQ ID NO:3 or a functional variant thereof.

As defined herein above, the term comprising refers to the option that further sequences may be present in addition to the specifically recited sequence. Such further sequences include, for example, a leader sequence, such as e.g. an oca-leader, a promoter as well as restriction enzyme—cloning sites for further alteration of the fusion protein, such as e.g. addition of further domains for extracellular presentation or for changing the affinity tag(s). The amino acid sequence of SEQ ID NO:3 may be encoded, for example, by the nucleic acid sequence of SEQ ID NO:4.

Most preferably, the fusion protein has the amino acid sequence as shown in SEQ ID NO:5. The amino acid sequence of SEQ ID NO:5 comprises all elements employed in the appended examples. Accordingly, this amino acid sequence may be used without further modifications when carrying out the present invention. The fusion protein having the amino acid sequence of SEQ ID NO:5 may for example be encoded by the nucleic acid sequence of SEQ ID NO:6.

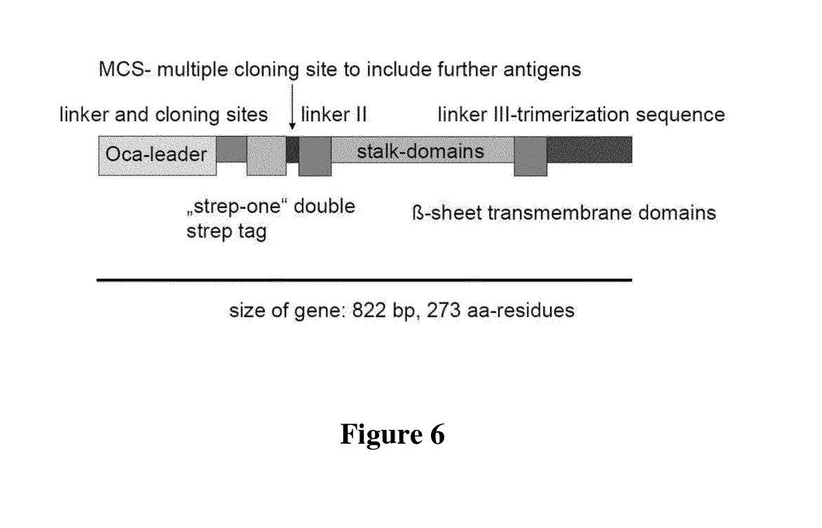

FIG. 6 provides an overview over the elements of a fusion protein comprising the amino acid sequence of SEQ ID NO:3 as well as the fusion protein having the amino acid sequence of SEQ ID NO:5.

In another preferred embodiment of the method of the invention, the genetic engineering comprises the introduction of at least one vector into the bacterial strain.

Means and methods of introducing nucleic acid sequences, such as e.g. vectors into bacterial cells have been described herein above. The term “at least one” is as defined herein above.

In another preferred embodiment of the method of the invention, the preparation of the strain-adapted vaccine in step (d) further comprises the addition of an adjuvant and/or a pharmaceutically acceptable carrier.

The term “adjuvant”, as used herein, is defined in accordance with the pertinent art and relates to a compound that enhances the recipient's immune response to the vaccine. Adjuvants are often added to promote an earlier, more potent response, and/or more persistent immune response to the vaccine, thereby allowing for a lower vaccine dosage. Non-limiting examples of adjuvants include e.g. aluminum hydroxide and aluminium phosphate, the organic compound Squalene but also compounds currently being tested or already qualified as adjuvants, such as e.g. QS21, Aluminum hydroxide and it's derivates, oil immersions, Lipid A and it's derivates (e.g. monophosphoryl lipid A (MPL), CpG motivs, Muramyldipeptid (MDP), Freund's Complete Adjuvant (FCA), Freund's incomplete Adjuvant (FIA) or MF59C (see e.g. Garçon and Van Mechelen. Recent clinical experience with vaccines using MPL- and QS-21-containing adjuvant systems. Expert Rev Vaccines. 2011 April; 10(4):471-86; Alving C R. Lipopolysaccharide, lipid A, and liposomes containing lipid A as immunologic adjuvants. Immunobiology. 1993 April; 187(3-5):430-46; Petrovsky and Aguilar. (2004). “Vaccine adjuvants: current state and future trends”. Immunol Cell Biol. 82 (5): 488-96; Weiner et al. (1997). Immunostimulatory oligodeoxynucleotides containing the CpG motif are effective as immune adjuvants in tumor antigen immunization. PNAS 94 (20): 10833-7; Yoo et al. Adjuvant activity of muramyl dipeptide derivatives to enhance immunogenicity of a hantavirus-inactivated vaccine. Vaccine. 1998 January-February; 16(2-3):216-24; Steiner et al. (1960) The local and systemic effects of Freund's adjuvant and its fractions. Archives of Pathology 70:424-434; U.S. Pat. No. 6,299,884 B, U.S. Pat. No. 6,451,325).

The term “pharmaceutically acceptable carrier” is defined in accordance with the pertinent art and relates to a non-toxic solid, semisolid or liquid filler, diluent, encapsulating material or formulation auxiliary of any type. Examples of suitable pharmaceutically acceptable carriers are well known in the art and include phosphate buffered saline solutions, water, emulsions, such as oil/water emulsions, various types of wetting agents, sterile solutions, organic solvents including DMSO etc. Compositions comprising such carriers can be formulated by well known conventional methods.

In accordance with this embodiment, an adjuvant and/or a pharmaceutically acceptable carrier is added during the vaccine preparation process, e.g. after purification and before administration of the vaccine (Petrovsky et al. Vaccine adjuvants: Current state and future trends; Immunology and Cell Biology (2004) 82, 488-496).

The present invention further relates to a strain-adapted vaccine obtainable by the method of the invention.

The vaccine may be in liquid, aerosolic, or solid form and may be, inter alia, in the form of (a) solution(s), (a) spray(s), (a) powder(s) or (a) tablet(s) or paste(s).

It will be appreciated that the vaccine, besides the active compound, may, optionally, comprise additional molecules capable of altering the characteristics of the active compound thereby. Such additional molecules are known in the art and include, without being limiting, adjuvants as described above, stabilisers, preservatives, pharmaceutically acceptable carriers, as described above, diluents and/or excipients. Stabilisers are employed to prevent alterations of the vaccine when exposed to e.g. heat, light, acidity or humidity. Non-limiting examples of often used stabilisers include monosodium glutamate (MSG) and 2-phenoxyethanol. Preservatives are typically added to prevent serious adverse side effects such as infection with bacteria or viruses grown in the vaccine during production or storage. Non-limiting examples of preservatives include antibiotics, formaldehyde, phenoxyethanol or thiomersal, which are usually added to vials of vaccine that contain more than one dose to prevent contamination and growth of potentially harmful bacteria/viruses. Conventional excipients that may be employed in the vaccine of the invention include binding agents, fillers, lubricants and various types of wetting agents. Vaccines comprising such additional molecules can be formulated by conventional methods. Adjuvants and pharmaceutically acceptable carriers have been defined herein above.

It will be appreciated that where a vaccine is provided without the addition of an adjuvant, then such an adjuvant may nonetheless be concomitantly administered with the vaccine or may be administered before or after the administration of the vaccine described herein.

The present invention further relates to a nucleic acid molecule encoding a fusion protein comprising a bacterial membrane protein fused to at least one affinity tag, wherein the bacterial membrane protein comprises or consists of the amino acid sequence of SEQ ID NO:1 or a functional variant thereof.

All of the definitions provided herein above with regard to the fusion protein for use in the method of the invention as well as nucleic acid molecules apply mutatis mutandis also the fusion protein of this embodiment.

Preferably, the nucleic acid molecule of the invention encodes a fusion protein as shown in SEQ ID NO: 3 or a functional variant thereof. More preferably, the nucleic acid molecule of the invention encodes a fusion protein of SEQ ID NO: 5 or a functional variant thereof.

The present invention also relates to a kit comprising: (a) the nucleic acid molecule of the invention; and (b) optionally an inhibitor of at least one protein of the Tol-Pal system family.

It will be appreciated that where the inhibitor in (b) is a peptide or protein inhibitor, said inhibitor may be present in the kit in proteinaceous form or as a nucleic acid molecule encoding said inhibitor for ease of introduction into bacteria.

The present invention further relates to a method for the preparation of bacterial-derived membrane vesicles, comprising the steps of (a) genetically engineering bacteria, wherein said genetic engineering comprises introducing a nucleic acid molecule encoding a first fusion protein, wherein the first fusion protein comprises a bacterial membrane protein fused to at least one affinity tag, (b) growing the genetically engineered bacterial strain obtained in step (a) in solution, (c) isolating membrane vesicles from the growth culture of step (b) by affinity purification using the affinity tag, (d) contacting the isolated membrane vesicles obtained (c) with a second fusion protein, wherein the second fusion protein comprises an antigen of interest fused to a binding partner for the at least one affinity tag present in the first fusion protein, and (e) formulating the membrane vesicles isolated in step (d) into a vaccine.

In accordance with this embodiment, the immune-mediating effect of bacterial membrane vesicles can be further enhanced, i.e. the immunogenicity of the bacterial-derived membrane vesicles can be improved. To this effect, bacteria are genetically engineered as described herein above. All of the definitions and preferred embodiment with regard to the method of the invention of preparing a strain-specific vaccine apply mutatis mutandis also this method of improving the immunogenicity of bacterial-derived membrane vesicles. For example, where appropriate, the formation of membrane vesicles may be enhanced by using an inhibitor of the Tol-Pal family system as described above.

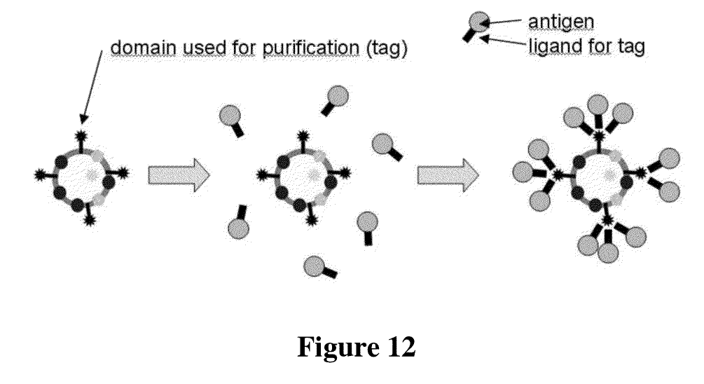

The isolated membrane vesicles are then contacted in step (d) with a second fusion protein. This second fusion protein comprises an antigen of interest fused to a binding partner for the at least one affinity tag present in the first fusion protein, as shown for example in FIG. 12. For example, where the at least one affinity tag in the first fusion protein is the Strep ONE tag, the corresponding binding partner in the second fusion protein may be the protein streptavidin or one of it's derivates such as strep-tactin.

The “antigen of interest” may be a protein which cannot be expressed in the bacteria producing the vesicles in the first place due to modifications which cannot be performed in bacteria, such as e.g. glycosylations or other posttranscriptional modifications. The antigen of interest may also be a protein that can be produced in larger quantities, higher purity or with better folding in another system or under different conditions than those chosen for vesicle purification.

It will be appreciated that the contacting in step (d) is carried out under conditions that result in the binding of the at least affinity tag to the corresponding binding partner present in the second fusion protein. Due to this binding, the antigen of interest becomes bound to the membrane vesicles and is efficiently presented to the immune system of the subject to be immunised with the resulting vaccine.

In accordance with this embodiment of the invention, purified membrane vesicles can additionally be used as vaccine carrier vehicle and mixed with any desired antigen coupled to an “adapter” sequence interacting with the purification tag, thereby enhancing the immune-stimulating effect of the membrane vesicles.

Preferably, the bacteria are strain-specific bacteria obtained from a sample from a patient as described herein above. Accordingly, it will be appreciated that this embodiment also encompasses a method for the preparation of a strain-adapted vaccine specific for a bacterial strain in accordance with the present invention, wherein prior to the step of formulating the isolated membrane vesicles into a vaccine, the membrane vesicles are contacted with a second fusion protein, wherein the second fusion protein comprises an antigen of interest fused to a binding partner for the at least one affinity tag present in the first fusion protein.

The definitions as well as the preferred embodiments provided herein above with regard to other embodiments such as e.g. the method for the preparation of a strain-adapted vaccine specific for a bacterial strain of the invention, the strain-adapted vaccine of the invention, the nucleic acid molecule of the invention, the kit of the invention etc. apply mutatis mutandis also to this embodiment relating to a method for the preparation of bacterial-derived membrane vesicles as outlined above. For example, preferred embodiments of the method for the preparation of a strain-adapted vaccine have their counterparts in preferred embodiments of the above defined method for the preparation of bacterial-derived membrane vesicles.

Unless otherwise defined, all technical and scientific terms used herein have the same meaning as commonly understood by one of ordinary skill in the art to which this invention belongs. In case of conflict, the patent specification, including definitions, will prevail.

The examples illustrate the invention:

### Example 1

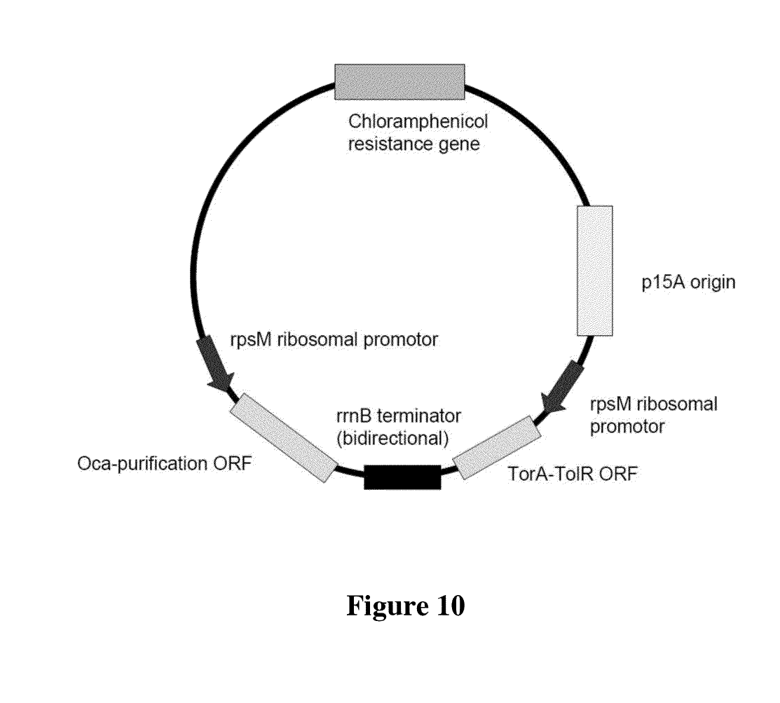

A well known E. coli (CFT073) isolate from a patient with pyelonephritis was washed with 10% glycerol for 7 times on ice and electroporated with the respective plasmid as depicted in FIG. 10.

Selection was performed on selective LB (luria-broth) agarose plates. Positive clones were cultivated o/n in 200 ml 37° C. LB broth including the plasmid's resistance antibiotic (Chloramphenicol 30 μg/ml). Bacteria were removed from the solution by centrifugation at 5000×g for 15 min at 4° C. The supernatant was subsequently sterile filtered (Millipore 0.22 μm Filter) using a vacuum pump and the sterile filtered supernatant was allowed to flow through a 1 ml Strep-Tactin® Superflow® column by gravity flow. The column was washed and eluted in accordance with the manufacturer's instructions with the supplied buffers. The vesicles eluted mainly in the 2^{nd} and 3^{rd} fraction. The eluate was checked for the presence of vesicles by SDS and Western Blot for the presence of trimeric strep tagged complexes (Strep-Tactin horseradish peroxidase (HRP) conjugate (2-1502-001) from iba-go according to the manufacturer's instructions). Eluate was stored at 4° C. in the fridge for two days to two weeks before administration.

Groups of seven weeks old Balb/C Mice were anaesthesized by Isofluran inhalation and 10 μl of the eluate was administered into each nostril using a shortened flexible “gel-loader” Tip. Doses were given on day 0 and day 5. Control animals were administered Eluate Buffer without vesicles in the same volume and frequency. 7 days after administration of the first dose, 50 μl of mouse blood was drawn through the lateral tail vein and serum was retrieved. 14 days after the start of vaccination, i.e. after administration of the first dose, a second blood sample was retrieved.

The amount of an IgM and an IgG response against the vesicle formulation used for vaccination was determined. Furthermore, the amount of the IgM and IgG response was also determined against a whole cell lysate of the pathogen used in this example, namely CFT073 wild type, the parent strain (without the respective plasmid).

For analysis, whole cell lysates and OMV vaccine preparations were subjected to one dimensional SDS PAGE and subsequent blot transfer onto nitrocellulose membranes. After blocking with 3% skimmed milk, mouse serum (with all inherent antibodies) was applied at a dilution of 1:1000 (3% BSA PBS) for 1 h at room temperature.

Bound serum antibodies were detected with the secondary antibodies Anti-IgM-HRPO-conjugate (produced in goat from sigma Aldrich) and Anti-IgG-HRPO-conjugate (produced in goat from sigma Aldrich) respectively. Secondary antibodies were diluted in 3% BSA PBS 1:10.000 and incubated for 1 h at room temperature. Visualization was performed with ECL subtrate (Pierce) according to the manufacturers instructions. The used protein standard was Fermentas PageRuler™pre-stained protein ladder.

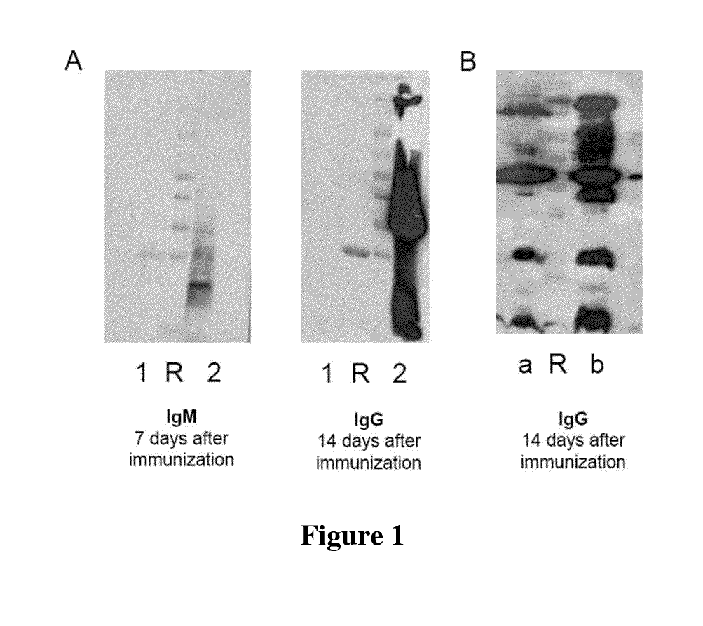

Analysis revealed strong titre increases against the OMV vaccine formulation and also against the whole bacterial cell, as shown in FIG. 1.

### Example 2

An avian pathogenic E. coli (APEC) which has been isolated from dead chicken was washed with 10% glycerol for 7 times on ice and electroporated with the respective plasmid as depicted in FIG. 10.

Selection was performed on selective LB (luria-broth) agarose plates. Positive clones were cultivated in 200 ml 37° C. LB broth including the plasmid's resistance antibiotic (Chloramphenicol 30 μg/ml). Bacteria were removed from the solution by centrifugation at 5000×g for 15 min at 4° C. and the supernatant was subsequently sterile filtered (Millipore 0.22 μm Filter) using a vacuum pump. The sterile filtered supernatant was allowed to flow through a 1 ml Strep-Tactin® Superflow® column by gravity flow. The column was washed and eluted in accordance with the manufacturer's instructions with the supplied buffers. The vesicles again eluted mainly in the 2^{nd} and 3^{rd} fraction. Eluate was checked for presence of vesicles by SDS and Western Blot for presence of trimeric strep tagged complexes (Strep-Tactin horseradish peroxidase (HRP) conjugate (2-1502-001) from iba-go according to the manufacturers instructions). Eluate was stored at 4° C. in the fridge for two days to two weeks before administration.

Groups of four Lohmann LSL chicken were inoculated with 50 μl of vaccine solution in total into the konjunctiva of the eyes (oculo-nasal application). This was performed with a regular Eppendorf reference pipette and plastic tip. Doses were given on day 0 and day 7. Control animals were administered Eluate Buffer without vesicles in the same volume and frequency. In parallel, a group of chicken was administered the same formulation intra muscularly. In all groups no adverse effects could be observed.

7 days after administration of the first dose, 2 ml of chicken blood was drawn and serum was retrieved. A second blood sample was prepared 21 days after the first administration.

For analysis, OMV vaccine preparations were subjected to one dimensional SDS PAGE and subsequent blot transfer onto nitrocellulose membranes. After blocking with 3% skimmed milk, chicken serum (with all inherent antibodies) was applied at a dilution of 1:500 (3% BSA PBS) for 1 h at room temperature.

Bound serum antibodies (IgY) were detected with the secondary antibody Anti-IgY-HRPO-conjugate (developed in rabbit). Secondary antibodies were diluted in 3% BSA PBS 1:10.000 and incubated for 1 h at room temperature. Visualization was performed with ECL subtrate (Pierce) according to the manufacturers instructions.

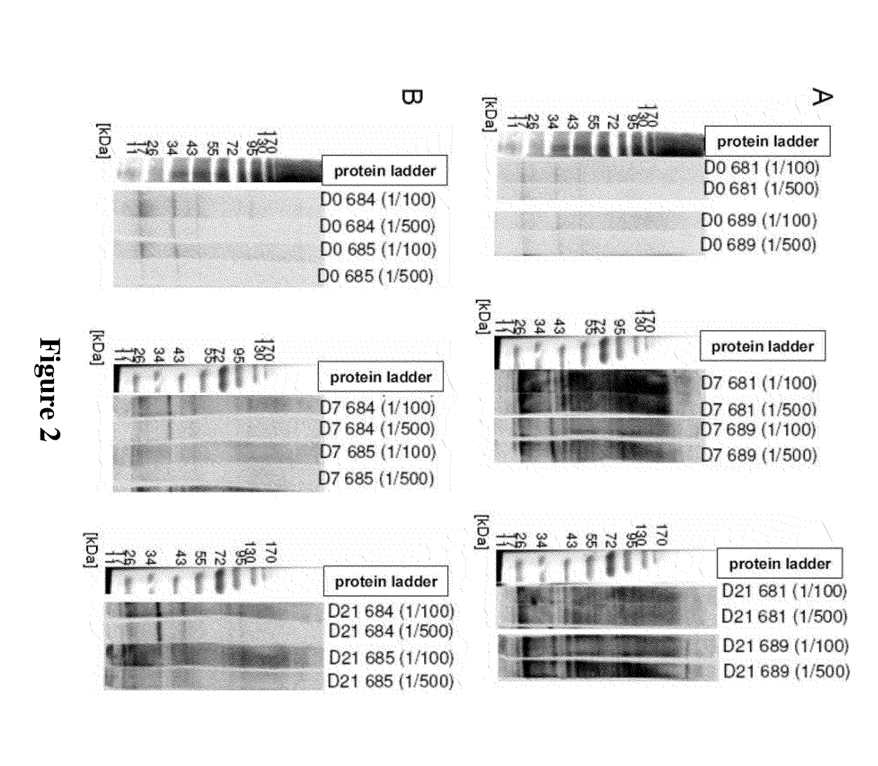

Analysis revealed strong titre increases against the OMV vaccine formulation in all vaccinated groups, whereby the intramuscular application seemed to be more efficient than the 50 μl oculonasal application, as shown in FIG. 2.

### Example 3

A human pathogenic Klebsiella pneumoniae was isolated from the lung, bloodstream and bones of a woman on ICU. The isolate was washed with 10% glycerol for 5 times on ice and electroporated with the respective plasmid. The organism was pan resistant with only Chloramphenicol being susceptible.

Selection was performed on selective LB (luria-broth) agarose plates. Positive clones were cultivated in 200 ml 37° C. LB broth including the plasmid's resistance antibiotic (Chloramphenicol 30 μg/ml). Bacteria were removed from the solution by centrifugation at 5000×g for 15 min at 4° C. and the supernatant was subsequently sterile filtered (Millipore 0.22 μm Filter) using a vacuum pump. The sterile filtered supernatant was allowed to flow through the 1 ml Strep-Tactin® Superflow® column by gravity flow. The column was washed and eluted in accordance with the manufacturer's instructions with the supplied buffers. The vesicles again eluted mainly in the 2^{nd} and 3^{rd} fraction. Eluate was checked for presence of vesicles by SDS and Western Blot for presence of trimeric strep tagged complexes (Strep-Tactin horseradish peroxidase (HRP) conjugate (2-1502-001) from iba-go according to the manufacturer's instructions). Eluate was stored at 4° C. in the fridge for two days to two weeks before administration.

The patient suffered from infection for 8 months with this highly aggressive organism although therapy was performed as good as possible with all available means.

Given the long survival (expected in such severe cases, in maximum therapy would be survival <1 week) the patient developed some sort of neutralizing immune response. To analyse this immune response, Western-Blot analysis was performed.

Serum (with all inherent antibodies) of the patient was blotted against the prepared putative vaccine as well as against whole cell extracts of the organism (for detailed protocol see Figure legend 3).

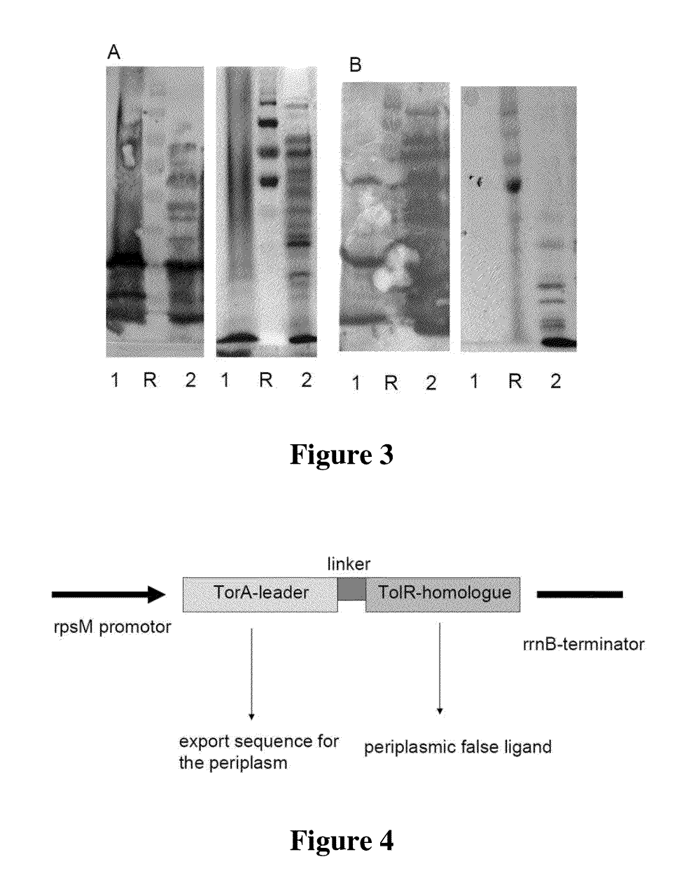

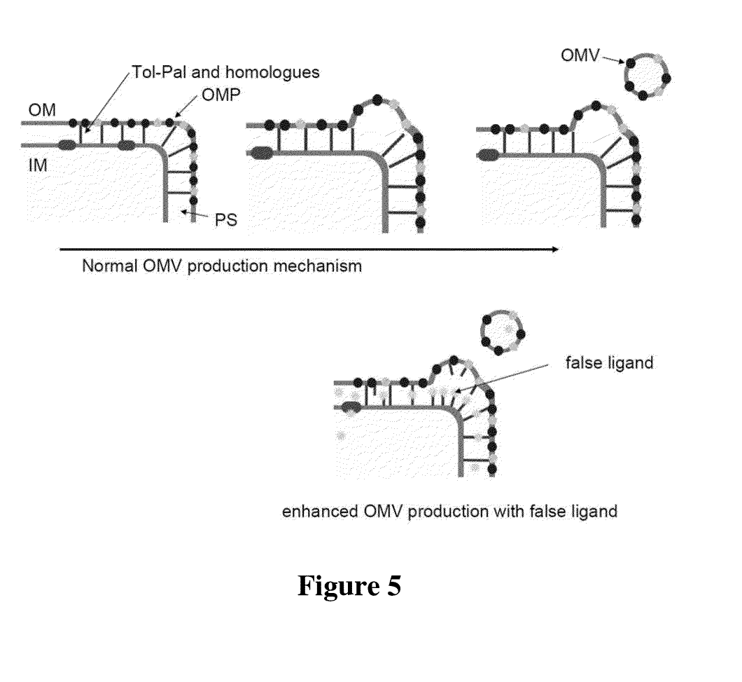

Immunoblots revealed Antibody titres >1:10.000 against even very low concentrations of OMV vaccine formulations and the whole, unchanged pathogenic organism. The response was pronounced against the OMV formulation when compared to the whole cell lysate, suggesting an enrichment of antigenic determinants on the vaccine formulation. (FIG. 3).

### Example 4

Other isolates retrieved from patients included Yersinia enterocolitica group O3, Salmonella enterica S enteritidis, Klebsiella pneumoniae.

These isolates were electroporated as described in Examples 1 to 3. Western Blot analysis proved expression and trimerization of the tag containing purification proteins. Purification was able to enrich OMV fractions in eluate fractions 2 to 4 according to the manufacturer's instructions (iba-go).

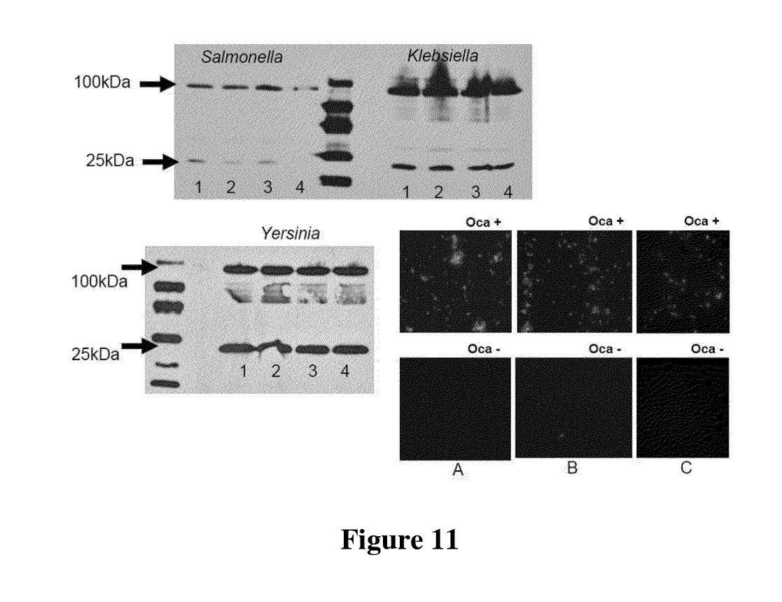

Fluorescence microscopy was performed on the isolates and proved extracellular localization of all the strep tags, as shown in FIG. 11.

### FURTHER REFERENCES

Alaniz, R. C., Deatherage, B. L., Lara, J. C. and Cookson, B. T.; Membrane vesicles are immunogenic facsimiles of Salmonella typhimurium that potently activate dendritic cells, prime B and T cell responses, and stimulate protective immunity in vivo. J. Immunol. 2007 Dec. 1; 179(11):7692-701. Erratum in: J. Immunol. 2008 Mar. 1; 180(5):3612.
Bakke, H., Lie, K., Haugen, I. L., Korsvold, G. E., Høiby, E. A., Meyer Næss, L, Hoist, J., Aaberge, I. S., Oftung, F. and Haneberg, B.; Meningococcal Outer Membrane Vesicle Vaccine Given Intranasally Can Induce Immunological Memory and Booster Responses without Evidence of Tolerance. Infect. Immun. 2001, 69:5010-5015.
Chen, D. J., Osterrieder, N., Metzger, S. M., Buckles, E., Doody, A. M., DeLisa, M. P. and Putnam, D.; Delivery of foreign antigens by engineered outer membrane vesicle vaccines. Proc Nat Acad Sci 2010:3099-3104.
Cotter, S. E., Surana, N. K. and St. Geme, J. W. 3^{rd}.; Trimeric autotransporters: a distinct subfamily of autotransporter proteins. Trends Microbiol. 2005 May; 13(5):199-205. Review.
Gurung, M., Moon, D. C., Choi, C. W., Lee, J. H., Bae, Y. C., Kim, J., Lee, Y. C., Seol, S. Y., Cho, D. T., Kim, S. I. and Lee, J. C.; Staphylococcus aureus Produces Membrane-Derived Vesicles That Induce Host Cell Death. PLoS ONE 2011, 6:e27958. doi:10.1371/journal.pone.0027958
Haneberg, B., Dalseg, R, Wedege, E., Høiby, E. A., Haugen, I. L., Oftung, F., Andersen, S. R., Meyer Næss, L, Aase, A., Michaelsen, T. E. and Holst, J.; Intranasal Administration of a Meningococcal Outer Membrane Vesicle Vaccine Induces Persistent Local Mucosal Antibodies and Serum Antibodies with Strong Bactericidal Activity in Humans. Infect. Immun. 1998, 66:1334-1341.
Henry T, Pommier S, Journet L, Bernadac A, Gorvel J P, Lloubès R.; Improved methods for producing outer membrane vesicles in Gram-negative bacteria. Res Microbiol. 2004 July-August; 155(6):437-46.
Ho J, Tambyah P A, Paterson D L.; Multiresistant Gram-negative infections: a global perspective. Curr Opin Infect Dis. 2010 December; 23(6):546-53. Review.
Hoist, J., Martin, D., Arnold, R., Campa Huergo, C., Oster, P., O'Hallahan, J. and Rosenqvist, E.; Properties and clinical performance of vaccines containing outer membrane vesicles from Neisseria meningitides. Vaccine 2009, 27S:B3-B12.
O'Fallon, E., Gautam, S, and D'Agata, E. M.; Colonization with multidrug-resistant gram-negative bacteria: prolonged duration and frequent cocolonization. Clin Infect Dis. 2009 May 15; 48(10):1375-81.
Park S. B., Jang H. B., Nho S. W., Cha I. S., Hikima J-i., Ohtani, M., Aoki, T. and Jung, T. S.; Outer Membrane Vesicles as a Candidate Vaccine against Edwardsiellosis. PLoS ONE 2011, 6: e17629. doi:10.1371/journal.pone.0017629
Rivera, J., Cordero, R. J. B., Nakouzi, A. S., Frases, S., Nicola, A. and Casadevall A.; Bacillus anthracis produces membrane-derived vesicles containing biologically active toxins. PNAS 2010, 107:19002-19007.
Roy K, Hamilton D J, Munson G P, Fleckenstein J M.; Outer membrane vesicles induce immune responses to virulence proteins and protect against colonization by enterotoxigenic Escherichia coli. Clin Vaccine Immunol. 2011 November; 18(11): 1803-8.

1-15. (canceled)

16. A method for the preparation of a strain-adapted vaccine specific for a bacterial strain, comprising the steps of:
(a) genetically engineering a bacterial strain obtained from a subject, wherein said genetic engineering comprises introducing a nucleic acid molecule encoding a fusion protein, wherein the fusion protein comprises a bacterial membrane protein fused to at least one affinity tag,
(b) growing the genetically engineered bacterial strain obtained in step (a) in solution,
(c) isolating membrane vesicles from the growth culture of step (b) by affinity purification using the affinity tag, and
(d) formulating the membrane vesicles isolated in step (c) into a strain-adapted vaccine.

17. A method for the preparation of bacterial-derived membrane vesicles, comprising the steps of:
(a) genetically engineering bacteria, wherein said genetic engineering comprises introducing a nucleic acid molecule encoding a first fusion protein, wherein the first fusion protein comprises a bacterial membrane protein fused to at least one affinity tag,
(b) growing the genetically engineered bacterial strain obtained in step (a) in solution,
(c) isolating membrane vesicles from the growth culture of step (b) by affinity purification using the affinity tag,
(d) contacting the isolated membrane vesicles obtained (c) with a second fusion protein, wherein the second fusion protein comprises an antigen of interest fused to a binding partner for the at least one affinity tag present in the first fusion protein, and
(e) formulating the membrane vesicles isolated in step (d) into a vaccine.

18. The method of claim 1, further comprising introducing an inhibitor of at least one protein of the Tol-Pal system family into the bacterial strain prior to step (c).

19. The method of claim 3, wherein the inhibitor of at least one protein of the Tol-Pal system family is a small molecule inhibitor, a ribozyme, an antisense construct, an antibody, a spiegelmer or an aptamer.

20. The method of claim 3, wherein the inhibitor of at least one protein of the Tol-Pal system family is selected from the group consisting of a soluble TolA and/or TolR periplasmic domain, the N-terminal domain of a group A colicin and the minor coat protein g3p.

21. The method of claim 3, wherein the at least one protein of the Tol-Pal system is/are selected from the group consisting of TolA, TolB, TolQ, TolR, Pal, Lpp, ybgC and NlpI.

22. The method of claim 1, wherein the bacterial strain is a Gram-negative or a Gram-positive bacterial strain.

23. The method of claim 7, wherein the Gram-positive bacterial strain is selected from the group consisting of Staphylococcus, Streptococcus, Enterococcus, Bacillus and Clostridium.

24. The method of claim 7, wherein the Gram-negative bacterial strain is selected from the group consisting of Escherichia, Enterobacter, Pseudomonas, Klebsiella, Stenotophomonas, Salmonella, Shigella, Yersinia and Acinetobacter.

25. The method of claim 1, wherein the bacterial membrane protein is selected from the group consisting of membrane pore-forming proteins, autotransporter proteins, receptor proteins and a protein comprising or consisting of the sequence of SEQ ID NO:1 or a functional variant thereof.

26. The method of claim 1, wherein at least one affinity tag is selected from the group consisting of a Strep-tag, chitin binding protein (CBP), maltose binding protein (MBP), glutathione-S-transferase (GST), FLAG-tag, HA-tag, Myc-tag, His-tag and derivatives thereof.

27. The method of claim 1, wherein the fusion protein comprises or consists of the amino acid sequence of SEQ ID NO:3 or a functional variant thereof.

28. The method of claim 2, further comprising introducing an inhibitor of at least one protein of the Tol-Pal system family into the bacterial strain prior to step (c).

29. The method of claim 13, wherein the at least one protein of the Tol-Pal system is/are selected from the group consisting of TolA, TolB, TolQ, TolR, Pal, Lpp, ybgC and NlpI.

30. The method of claim 2, wherein the bacterial membrane protein is selected from the group consisting of membrane pore-forming proteins, autotransporter proteins, receptor proteins and a protein comprising or consisting of the sequence of SEQ ID NO:1 or a functional variant thereof.

31. The method of claim 1, wherein at least one affinity tag is selected from the group consisting of a Strep-tag, chitin binding protein (CBP), maltose binding protein (MBP), glutathione-S-transferase (GST), FLAG-tag, HA-tag, Myc-tag, His-tag and derivatives thereof.

32. The method of claim 2, wherein the fusion protein comprises or consists of the amino acid sequence of SEQ ID NO:3 or a functional variant thereof.

33. A strain-adapted vaccine obtainable by the method of claim 1.

34. A nucleic acid molecule encoding a fusion protein comprising a bacterial membrane protein fused to at least one affinity tag, wherein the bacterial membrane protein comprises or consists of the amino acid sequence of SEQ ID NO:1 or a functional variant thereof.

35. A kit comprising:
(a) the nucleic acid molecule of claim 19; and
(b) optionally, an inhibitor of at least one protein of the Tol-Pal system family.


SEQUENCE LISTING (104 sequences)

SEQ ID NO: 1 - protein, 107 aa, artificial sequence
  stem domain
AEVLGIANNYTDSKSAETLENARKEAFDLSNDALDMAKKHSNSVARTTLETAEEHTNKKSAETLASANVYADSKSSHTLKTANSYTDVTVSNSTKKAIRESNQYTDH

SEQ ID NO: 2 - DNA, 321 bp, artificial sequence
  stem domain
gcagaagttctgggcattgccaataactataccgatagcaaaagtgcagaaaccctggaaaatgcacgtaaagaagcatttgatctgagcaatgatgcactggatatggccaaaaaacatagcaatagcgttgcacgtaccacactggaaaccgcagaagaacataccaacaaaaaatctgccgaaacactggcaagcgctaacgtttacgctgactctaaatcttctcacaccctgaaaaccgctaactcttacaccgacgttaccgtttctaactctaccaaaaaagctatccgtgaatctaaccagtacaccgaccac

SEQ ID NO: 3 - protein, 248 aa, artificial sequence
  amino acid sequence of artificial membrane protein
NNESAWSHPQFEKGGGSGGGSGGSAWSHPQFEKGGGSGTGELKTQENANKKSAEVLGIANNYTDSKSAETLENARKEAFDLSNDALDMAKKHSNSVARTTLETAEEHTNKKSAETLASANVYADSKSSHTLKTANSYTDVTVSNSTKKAIRESNQYTDHKFHQLDNRLDKLDTRVDKGLASSAALNSLFQPYGVGKVNFTAGVGGYRSSQALAIGSGYRVNESVALKAGVAYAGSSDVMYNASFNIEW

SEQ ID NO: 4 - DNA, 747 bp, artificial sequence
  nucleic acid sequence of artificial membrane protein
aacaacgaaagcgcatggtcacatccgcagtttgaaaaaggtggtggtagcggtggtggttcaggtggtagtgcttggagccaccctcagttcgagaaaggcggtggcagtggtaccggtgagctcaaaacccaagaaaatgcaaacaaaaaaagcgcagaagttctgggcattgccaataactataccgatagcaaaagtgcagaaaccctggaaaatgcacgtaaagaagcatttgatctgagcaatgatgcactggatatggccaaaaaacatagcaatagcgttgcacgtaccacactggaaaccgcagaagaacataccaacaaaaaatctgccgaaacactggcaagcgctaacgtttacgctgactctaaatcttctcacaccctgaaaaccgctaactcttacaccgacgttaccgtttctaactctaccaaaaaagctatccgtgaatctaaccagtacaccgaccacaaatttcaccagctggacaaccgtctggacaaactggacacccgtgttgacaaaggtctggcttcttctgctgctctgaactctctgttccagccgtacggtgttggtaaagttaacttcaccgctggtgttggtggttaccgttcttctcaggctctggctatcggttctggttaccgtgttaacgaatctgtggctctgaaagctggtgttgcttacgctggttcttctgacgttatgtacaacgcttctttcaacatcgaatggtaa

SEQ ID NO: 5 - protein, 273 aa, artificial sequence
  amino acid sequence of fusion protein without promoter, with Oca leader
MTKDFKISVSAALISALFSSPYAFANNESAWSHPQFEKGGGSGGGSGGSAWSHPQFEKGGGSGTGELKTQENANKKSAEVLGIANNYTDSKSAETLENARKEAFDLSNDALDMAKKHSNSVARTTLETAEEHTNKKSAETLASANVYADSKSSHTLKTANSYTDVTVSNSTKKAIRESNQYTDHKFHQLDNRLDKLDTRVDKGLASSAALNSLFQPYGVGKVNFTAGVGGYRSSQALAIGSGYRVNESVALKAGVAYAGSSDVMYNASFNIEW

SEQ ID NO: 6 - DNA, 822 bp, artificial sequence
  nucleic acid sequence of fusion protein without promoter, with Oca leader
atgaccaaagacttcaaaatctctgtttctgctgctctgatctctgctctgttctcttctccgtacgctttcgctaacaacgaaagcgcatggtcacatccgcagtttgaaaaaggtggtggtagcggtggtggttcaggtggtagtgcttggagccaccctcagttcgagaaaggcggtggcagtggtaccggtgagctcaaaacccaagaaaatgcaaacaaaaaaagcgcagaagttctgggcattgccaataactataccgatagcaaaagtgcagaaaccctggaaaatgcacgtaaagaagcatttgatctgagcaatgatgcactggatatggccaaaaaacatagcaatagcgttgcacgtaccacactggaaaccgcagaagaacataccaacaaaaaatctgccgaaacactggcaagcgctaacgtttacgctgactctaaatcttctcacaccctgaaaaccgctaactcttacaccgacgttaccgtttctaactctaccaaaaaagctatccgtgaatctaaccagtacaccgaccacaaatttcaccagctggacaaccgtctggacaaactggacacccgtgttgacaaaggtctggcttcttctgctgctctgaactctctgttccagccgtacggtgttggtaaagttaacttcaccgctggtgttggtggttaccgttcttctcaggctctggctatcggttctggttaccgtgttaacgaatctgtggctctgaaagctggtgttgcttacgctggttcttctgacgttatgtacaacgcttctttcaacatcgaatggtaa

SEQ ID NO: 7 - protein, 392 aa, Salmonella enteritidis
MSKATEQNDKLKRAIIISAVLHIILFAVLIWSSFDEHIEASAGGGGGSAIDAVMVDPGAVVQQYNRQQDQQASARRAEEERKKLQQQQAEELQQKQAAEQERLKQLEKERLAAQEQQKQAEEAAKLAQQQQQQAEEAAKAAADAKKKAEAEAAKAAADAKKKAEAEAAKAAADAKKKAEAEAVKAAADAKKKAEAEAAKAAAEAKKKAEAEAAKAAAEAKKKADAEAAKAAAEAKKKADAAAAKAAADAKKKAAAEKAAAAEGVDDLLGDLSSGKNAPKTGGGAKGNGQPSKDSGTSGANGGATGADISAYAKQIQVAIQSRLYDASLYQGKQCVLHISLAPDGSLKSITSEGGDPALCQAALMAAKTAKIPKPPSQAVYEKIKDAKLDFKL

SEQ ID NO: 8 - protein, 124 aa, Acinetobacter calcoaceticus
MKNFIIVASLFLLVGCATGHKQVTPTQVADNKIISEAEKYKKQYQKKIYKTWDIPKSSTGMSASVKVFLTDTGEIEQIIFLDKEEQKFKTSIEKAIWHSSPFVLPSNPEVRKQARKFNIKFQVK

SEQ ID NO: 9 - protein, 386 aa, Yersinia enterocolitica subsp. enterocolitica 8081
MWKSSVGKATEQNDKLNRAVIVSVVLHIILIALLIWGSLTQTTEMGGGGAGGEVIDAVMVDPGAVTEQYNRQQQQQTDAKRAEQQRQKKAEQQAEELQQKQAAEQQRLKELEKERLQAQEDAKQAAEEQKKQAAEQQKEAAEQQKAAAAAAAKAKEEQKQAEAAAAQAKAEADKLVKAQAEAQKKAEAEAKKEAAVAAAAKKQADADAKKAADAAEKAATAAAEKKAAADAEKKAAADAKKAAAAEAKKKAAAEASAATDVDDLFGGLANSKNAPKSGSGAGAAAAGKGGGKKSGASAGDISGYLGQVTAAIQSKFYDADLYKGRTCNLRIKLAPDGLLIDVKQEGGDPALCQAAIAAAKLAKIPKPPSQDVYEVFKNAPLVFKPQ

SEQ ID NO: 10 - protein, 421 aa, Shigella dysenteriae Sd197
MSKAIEQNDKLKRAIIISAVLHVILFAALIWSSFDENIEASAGGGGGSSIDAVMVDSGAVVEQYKRMQSQESSAKRSDEQRKMKEQQAAEELREKQAAEQERLKQLEKERLAAQEQKKQAEEAAKQAELKQKQAEEAAAKAAADAKAKAEADAKAAEEAAKKAAADAKKKAEAEAAKAAAEAQKKAEAAAAALKKKAEAAEAAAAEARKKAATEAAEKAKAEAEKKAAAEKAAADKKAAAEKAAADKKAAEKAAAEKAAADKKAAAEKAAADKKAAAAKAAAEKAAAAKAAAEADDIFGELSSGKNAPKTGGGAKGNNASLAGSGNTKNNGASGADINNYAGQIKSAIESKFYDASSYAGKTCTLRIKLAPDGMLLDIKPEGGDPALCQAALAAAKLAKIPKPPSQAVYEVFKNAPLDFKP

SEQ ID NO: 11 - protein, 376 aa, Salmonella enterica subsp. enterica serovar Typhi str. P-stx-12
MSKATEQNDKLKRAIIISAVLHIILFAVLIWSSFDEHIEASAGGGGGSAIDAVMVDPGAVVQQYNRQQDQQASARGAEEERKKLQQQQAEELQQKQAAEQERLKQLEKERLAAQEQQKQAEEAAKLAQQQQQAEEAAKAAADAKKKAEAEAAKAAADAKKKAEAEAAKAAADAKKKAEAEAAKAAAEAKKKAEAEAAKAAADAKKKADAEAAKAAAEAKKKADAAAAKAAADAKKKAAAEKAAAAEGVDDLLGDLSSGKNAPKTGGGAKGNGQPSKDSGTSGANGGTTGADISAYAKQIQVAIQSRLYDASLYQGKQCVLHISLAPDGSLKSITSEGGDPALCQAALMATKTAKIPKPPSQAVYEKIKDAKLDFKL

SEQ ID NO: 12 - protein, 332 aa, Pseudomonas stutzeri group A1501
MQQRDSSPSQSYFWPTVLAVGLHVIIFGMLFVSFSMTPELPPSKPIVQATLYQLKSQSQATTQTNQKIAGEAKKTAAKQYEAEQMEQRKIEQQKQAAAKAAEQKKAEEARKAEAAKAAADKTAAAKKADEAKKVEQQKQAEIAKKKAAEELAKKKAAEEAKKKAAEEAKKKAAEEAKKKAAAEAAKKKAAEEAKKKAAAEAARKAAEDKKAQALAELLSDTTERQQAMADTHGDQVAGNFDDLIRLRAAEGWTRPPSARNNMTVQLQVNMLPDGTITNVSVSRSSGDVPFDNSAVAAVKNIGRLTEMQGLSPQEFQPYRSFKMTFTPEDLAL

SEQ ID NO: 13 - protein, 347 aa, Pseudomonas aeruginosa LESB58
MKQQFERSPSESYFWPVVLAVVLHVLIFAMLFVSWAFAPELPPSKPIVQATLYQLKSKSQATTQTNQKIAGEAKKTASKQYEVEQLEQKKLEQQKLEQQKLEQQQVAAAKAAEQKKADEARKAEAQKAAEAKKADEAKKAAEAKAAEQKKQADIAKKRAEDEAKKKAAEDAKKKAAEDAKKKAAEEAKKKAAAEAAKKKAAVEAAKKKAAAAAAAARKAAEDKKAQALAELLSDTTERQQALADEVGSEVTGSLDDLIVNLVSQQWRRPPSARNGMSVEVLIEMLPDGTITNASVSRSSGDKPFDSSAVAAVRNVGRIPEMQQLPRATFDSLYRQRRIIFKPEDLSL

SEQ ID NO: 14 - protein, 441 aa, Klebsiella oxytoca KCTC 1686
MSKATEQNDKLKRAIIVSVALHIVLIALLIWSSFDEHLDASAGGGGGSSIDAVMVDPGAVVKNYDRQQQQQASARRAAEQREKQAQQQAEELREKQAAEQERLKQLEQDRLQAQEAAKEAKEQQKQAEEAAAKAAAAAKAKADAQAKEAQEAAAKATADAKAKADAQAKAAEAAAAKAAADAKKQAEAEAAKAAADAQKKAEADAAKKAQQEAEKKAQQQAEKKAQQEAAKQAAAEKAAAEKAAEKAAAQKAAAEKAAAEKAAAAEKAAAEKAAAAKAAAAEKAAADKAAKAAAAKAAAAKKAAAAKEANGVDDLLGDLSSGKNAPKGNTSGSAGGSSPAKGSKQGQGGASQAEINSYISQVQAAIKGHLYDWDTYKGKTCTLRVNLAQDGTLLSVKSEGGDPAFCQQMLAATSRMTKFPKPPSQAVYETFKNAPLDFKPQ

SEQ ID NO: 15 - protein, 167 aa, Enterobacter cloacae SCF1 chromosome
MDDLPSFRTEIHLADYLTEEEDTLSFPNKIMTVMKKIILIAICLVSGCTGMSHSRDDVKAAAPERGPYAGKTSHTAPVITPAVSSYAAAVRDEITKHFFDVKRYRGKVCSLRLSMERNGTVNSVNTTGGDPKVCEAAVNAVKLSTLPPVPDEQTYKVFKNVALDFTP

SEQ ID NO: 16 - protein, 332 aa, Pseudomonas stutzeri ATCC 17588
MQQRDSSPSQSYFWPTVLAVGLHVIIFGMLFVSFSMTPELPPSKPIVQATLYQLKSQSQATTQTNQKIAGEAKKTAAKQYEAEQMEQRKIEQQKQAAAKAAEQKKAEEARKAEAAKAAADKAAAARKAEEAKKVEQQKQAEIAKKKAAEELAKKKAAEEAKKKAAEEAKKKAAEEAKKKAAAEAAKKKAAEEAKKKAAAEAARKAAEDKKAQALAELLSDTTERQQAMADTHGDQVAGNFDDLIRLRAAEGWTRPPSARNNMTVQLQVNMLPDGTITNVSVSRSSGDVPFDNSAVAAVKNIGRLTEMQGLSPQEFQPYRSFKMTFTPEDLAL

SEQ ID NO: 17 - protein, 413 aa, Shigella flexneri 2a str. 2457T
MSKATEQNDKLKRAIIISAVLHVILFAALIWSSFDENIEASAGGGGGSSIDAVMVDSGAVVEQYKRMQSQESSAKRSDEQRKMKEQQAAEELREKQAAEQERLKQLEKERLAAQEQKKQAEEAAKQAELKQKQAEVAAAKAAADAKAAEEAAKKAAADAKKKAEAEAAKAAAEAQKKAEVAAAALKKKAEAAEAAAAEARKKAATEAAEKAKAEAEKKAAAEKAAADKKAAAEKAAADKKAAEKAAADKAAADKKAAAEKAAADKKAAAAKAAAEKAAAAKAAAEADDIFGELSSGKNAPKTGGGAKGNNASPAGSGNTKNNGASGADINNYAGQIKSAIESKFYDASSYAGKTCTLRIKLAPDGMLLDIKPEGGDPALCQAALAAAKLAKIPKPPSQAVYEVFKNAPLDFKP

SEQ ID NO: 18 - protein, 395 aa, Shigella sonnei Ss046
MSKATEQNDKLKRAIIISAVLHVILFAALIWSSFDENIEASAGGGGGSSIDAVMVDSGAVVEQYKRMQSQESSAKRSDEQRKMKEQQAAEELREKQAAEQERLKQLEKERLAAQEQKKQAEEAAKQAELKQKQAEEAAAKAAADAKAKAEADAKAKAEADAKAAEEAAKKAAADAKKKAEAEAAKAAAQKKAEAAAAALKKKAEAAEAAAAEARKKAATEAAEKAKAEAEKKAAAEKAAADKKAAEKAAAEKAAADKKAAAEKAAAEADDIFGELSSGKNAPKTGGGAKGNNASPAGSGNTKNNGASGADINNYAGQIKSAIESKFYDASSYAGKTCTLRIKLAPDGMLLDIKPEGGDPALCQAALAAAKLAKIPKPPSQAVYEVFKNAPLDFKP

SEQ ID NO: 19 - protein, 395 aa, Shigella boydii; identical to SEQ ID NO: 18

SEQ ID NO: 20 - protein, 253 aa, Acinetobacter baumannii group AB307-0294
MKMMKTAIVTASVLASASIFAQSAGVNAGASAQVNVQPGGLVSGVANTVKNTAHTVGNTAKHAGHVAADTTVKATKKTTGKVTELSSKAATGTKHVASEAVTGTKHFATEAATGTKNLATKAATGTKNLAVEAKADTKAHLDAVKTKVAEKQADQKEFTAEKQADAQARVDAVKARVAQNQAEQKEFVADTKADAQAKLNTAQPAHGVNAQTGVNVGVNVAGINANANVNAGAQASTQKGEKKSFIKGLFGTN

SEQ ID NO: 21 - protein, 441 aa, Klebsiella pneumoniae
MSKATEQNDKLKRAIIISVALHIILIALLIWSSFDEHLDASAGGGGGSSIDAVMVDPGAVVNNYNRQQQQQASARRAAEQREKQAQQQAEELREKQAAEQERLKQLEQERLQAQEAAKEAKEQQKQAEEAAAKAAAAAKAKADAQAKEAQEAAAKAAAEAKAKADAQAKAAEQAAAKAAADAKKQAEAAAAKAAAEAKKQAEAEAAKAAAEAQKKAEAAAAKKAQQEAEKKAQQEAAKQAAAEKAAAEKAAEKAAAQKAAAEKAAAEKAAAAEKAAAAKAAAAEKAAADKAAKAAAAKAAAAKKAAAAKEADGVDNLLGDLSSGKNAPKTGGGAKGNNASAAGSGNTKNSASGADINNYAGQIKSAIESKFYDASSYAGKTCTLRIKLAPDGLLLNIQSEGGDPALCQAALAAARQAKFPKPPSQAVYEVFKNAPLDFKPQ

SEQ ID NO: 22 - protein, 539 aa, Salmonella enterica subsp. enterica serovar Paratyphi B str. SPB7
MAIFGSGIMFRQGKFVFLIQRSDDGTWCQPGGTVEQGELAIDAARREVLEEVGYQYDGPLIPHSVHGDYLTFRAEVPERFEAKLNDESLAAGWFHIDDLPKPLHQPFAEMLARQALNETEVAALIADGTLSSPQFFINMWMFAIRVTGTGVTWRSADQQMAFRNPDDYLTPEFLQRVAGLPLIWLHPEKNTLDSNEFAKRVIGTLTNSWVADNGEVWAIARVYDAEAAEIMATRQLSTSPTVKFVEVPDSIIEIDGQPLLVEPSPELLDHVAICEQGVWDKLLAPTGVKSDSIPNEAEKMDEEKIVALINKAIDARMAKADSEAADLKAKADAEEAAKKEKADAEEKEAEEAKAKADAEEKAAKEKADAEAKEKADTEEAERMAKEKADADVRREIAELKSRIPTELSDEERNEVADAQVKADSVFSCFGKRAPVPLSGEKPLAYRRRLMIQLQEHSPDFKTVDLSSIADSALLSVAEKTIYADAQKSAILSVGPGMLREIKRADATGRQISTFEGDPAVTWAPFQSGKRQVTSFNNQA

SEQ ID NO: 23 - protein, 307 aa, Francisella sp.
MANLNYQKVITFCKKTVDENPFLVKAILVHVGIVILLYTLAFISDLKFETSQAALSAQVEKAPKKLQIIQATSISSSELNKQLSAYENHQQELKQAREDIKQAKRQALIKHQQKLKAQAEAEKRAKLEAKRKAILEAKKKAQAEAKRQAEQKRQAQEEAERKKELEEKKKAQEEAKRKKEQELKAKQEAEEKARQERLAKARAEAIASAKREAAKNQADLAVSKYVTEYTDRVGSNWIKDDCRGIYDLPRAIIRNGKFIRLTGTSGDYGCDQSLIDAIKNTTPPDISNPVARRTIQTENISFIFKQS

SEQ ID NO: 24 - protein, 368 aa, Pseudomonas putida group
MQQREPSASESYFWPSIWAIGLHVLVFALLFVSFAMTPELPPSKPIVQATLYQLKSKSQATTQTNQKIAGEAKKTASRQTEVEQLEQKKVEQEAVKAAEQKKADAAQKAEEAREAAEAKKAEAAAEAAKAAEAKKAAEAKKAEEAKKAAEKQQADIAKKKAEEEKKKAEEEAKKEAAEEAKKQAAEDAKKKAAEEAKKKAAEDAKKKAVAEEAKKKAAEEAKKKAAADAQKKKAQEAARKAAEDKKAQALAELLSDTTERQQALADEQGDQVAGDFDDLIRMRAAEGWARPPSARKGMTVVLQINMLPDGTITSVSVARSSGDGPYDSSAVAAVKNIGRLTEMQGMKPSDFNQYRSFKMTFTPEDLAL

SEQ ID NO: 25 - protein, 391 aa, Yersinia pseudotuberculosis
MWKSSVGKATEQNDKLNRAVIVSVVLHIILIALLIWGSLTQTTEMGGGGAGGEVIDAVMVDPGAVTEQYNRQQQTDAKRAEQQRQKKAEQQAEELQQKQAAEQQRLKELEKERLQAQEDAKLAAEEQKKQVAEQQKQIAEQQKQAAEQQKIAAAAVAKAKEEQKQAETAAAQAKAEADKIVKAQAEAQKKAEAEAKKEAAVAAAAKKQADADAKKAVEVAEKAAADAAEKKAAADAEKKAAAAKKVAAAAEAKKKAAAEAAASTDVDDLFGGLANAKNAPKSGSGAGAAAAGKGGGKKSGASGADISGYLGQITGAIQSKFYDADLYKGRTCDLRIKLAPDGLLIDVKAEGGDPALCQAAIAAAKQAKIPKPPSTDVYEQFKNAPLVFKPQ

SEQ ID NO: 26 - protein, 351 aa, Stenotrophomonas maltophilia R551-3
MHADALPPPHQREPGWGLPLALAVLVHLLVALIFIGAWLWSPQRNTDAAAGDPSVEASLALSSSEAAAARQALRQSEKLEDLPPPVAEPIPVPEDTIPPPQPIPEPRPQDAPTPQQQQAQERVAQPDTKDQDAVSALAISQEKAKQEQEAKRRQEQIDLTERKRQEEAEQKLRLAKQQEADEKKKQAEQERVAADKAEAEKQKKIAEIRARREQAEKEAKLAEQKLRQVAAARNAAGSAAAGASGAAQPAGGGGGTSDDLSAKYAAAIQAKVLSQWVRPDTVPLGQRCTITITQIPGGTVMQAKVSPNCPYDEAGRRSIEAAVLNAQPLPYRGFESVFARTLNFTFTAQDR

SEQ ID NO: 27 - protein, 388 aa, Yersinia pestis
MGKATEQNDKLNRAVIVSVVLHIILIALLIWGSLTQTTEMGGGGAGGEVIDAVMVDPGAVTEQYNRQQQQQTDAKRAEQQRQKKAEQQAEELQQKQAAEQQRLKELEKERLQAQEDAKLAAEEQKKQVAEQQKQIAEQQKQAAEQQKIAAAAVAKAKEEQKQAETAAAQAKAEADKIVKAQAEAQKKAEAEAKKEAAVAAAAKKQADADAKKAVEVAEKAAADAAEKKAAADAEKKAAAAKKVAAAAEAKKKAAAEAAASTDVDDLFGGLANAKNAPKSGSGAGAAAAGKGGGKKSGASGADISGYLGQITGAIQSKFYDADLYKGRTCDLRIKLAPDGLLIDVKAEGGDPALCQAAIAAAKQAKIPKPPSTDVYEQFKNAPLVFKPQ

SEQ ID NO: 28 - protein, 359 aa, Pseudomonas fluorescens group
MQQQREPSASESYFWPSVLAIGLHVLVFGMLFVSFAMTPELPPAKPIVQATLYQLKSKSQATTQTNQKLAGEAKKSAARQTEVEQMEQKKVEQEAIKAAEQKKEEAAQKAEEAKKADEAKKADEAKKADEAKKADEAKKTAEAKKAEEKQLADIAKKKAEEEAKKAAEEEAKKAAAEEAKKKIVEDAKKKAAEDAKKKAEAEEAKKKVAEDAKKKAAADAAKKKAQDAARKSAEDKKAQALADLLSDTPERQQALADEQGDEVAGSYDDLIRARAAEGWARPPSARKGMTVVLQIGMLPDGTVTSVSVAKSSGDGPFDASAVAAVKNIGRLTEMQGMKPSDFAPYRSFKMTFTPEDLAL

SEQ ID NO: 29 - protein, 268 aa, Acinetobacter baumannii
MKYLTKRVYSLKDIAMKMMKTAIVTASVLASASIFAQSAGVNAGASAQVNVQPGGLVSGVANTVKNTAHTVGNTAKHAGHVAADTTVKATKKTTGKVTELSSKAATGTKHVASEAVTGTKHFATEAATGTKNLATKAATGTKNLAVEAKADTKAHLDAVKTKVAEKQADQKEFTAEKQADAQARVDAVKARVAQNQAEQKEFVADTKADAQAKLNTAQPAHGVNAQTGVNVGVNVAGINANANVNAGAQASTQKGEKKSFIKGLFGTN

SEQ ID NO: 30 - protein, 407 aa, Salmonella enterica subsp. enterica serovar Typhimurium str. SL1344
MSKATEQNDKLKRAIIISAVLHIILFAVLIWSSFDEHIEASAGGGGGSAIDAVMVDPGAVVQQYNRQQDQQASARRAEEERKKLQQQQAEELQQKQAAEQERLKQLEKERLAAQEQQKQAEEAAKLAQQQQQQAEEAAKAAADAKKKAEAEAAKAAADAKKKAEAEAVKAAADAKKKAEAEAAKAAADAKKKAEAEAAKAAAEAKKKAEAEAAKAAAEAKKKADAEAAKAAAEAKKKADAAAAKAAAEAKKKADAAAAKAAADAKKKAAAEKAAAAEGVDDLLGDLSSGKNAPKTGGGAKGNGQPSKDSGTSGANGGATGADISAYAKQIQVAIQSRLYDASLYQGKQCVLHISLAPDGSLKSITSEGGDPALCQAALMAAKTAKIPKPPSQAVYEKIKDAKLDFKL

SEQ ID NO: 31 - protein, 97 aa, Escherichia coli
GNTKNNGASGADINNYAGQIKSAIESKFYDASSYAGKTCTLRIKLAPDGMLLDIKPEGGDPALCQAALAAAKLAKIPKPPSQAVYEVFKNAPLDFKP

SEQ ID NO: 32 - protein, 97 aa, Enterobacter cloacae
KTSHTAPVITPAVSSYAAAVRDEITKHFFDVKRYRGKVCSLRLSMERNGTVNSVNTTGGDPKVCEAAVNAVKLSTLPPVPDEQTYKVFKNVALDFTP

SEQ ID NO: 33 - protein, 97 aa, Shigella flexneri; identical to SEQ ID NO: 31

SEQ ID NO: 34 - protein, 97 aa, Shigella sonnei; identical to SEQ ID NO: 31

SEQ ID NO: 35 - protein, 94 aa, Shigella boydii
GNTKNNGASGADINNYAGQIKSAIESKFYDASSYAGKTCTLRIKLAPDGMLLDIKPEGGDPALCQAALAAAKLAKIPKPPSQAVYEVFKNAPLD

SEQ ID NO: 36 - protein, 97 aa, Shigella dysenteriae; identical to SEQ ID NO: 31

SEQ ID NO: 37 - protein, 110 aa, Pseudomonas aeruginosa
SDTTERQQALADEVGSEVTGSLDDLIVNLVSQQWRRPPSARNGMSVEVLIEMLPDGTITNASVSRSSGDKPFDSSAVAAVRNVGRIPEMQQLPRATFDSLYRQRRIIFKP

SEQ ID NO: 38 - protein, 109 aa, Pseudomonas putida
SDTTERQQALADEQGDQVAGDFDDLIRMRAAEGWARPPSARKGMTVVLQINMLPDGTITSVSVARSSGDGPYDSSAVAAVKNIGRLTEMQGMKPSDFNQYRSFKMTFTP

SEQ ID NO: 39 - protein, 109 aa, Pseudomonas fluorescens
SDTPERQQALADEQGDEVAGSYDDLIRARAAEGWARPPSARKGMTVVLQIGMLPDGTVTSVSVAKSSGDGPFDASAVAAVKNIGRLTEMQGMKPSDFAPYRSFKMTFTP

SEQ ID NO: 40 - protein, 109 aa, Pseudomonas stutzeri
SDTTERQQAMADTHGDQVAGNFDDLIRLRAAEGWTRPPSARNNMTVQLQVNMLPDGTITNVSVSRSSGDVPFDNSAVAAVKNIGRLTEMQGLSPQEFQPYRSFKMTFTP

SEQ ID NO: 41 - protein, 102 aa, Klebsiella pneumoniae
ASAAGSGNTKNSASGADINNYAGQIKSAIESKFYDASSYAGKTCTLRIKLAPDGLLLNIQSEGGDPALCQAALAAARQAKFPKPPSQAVYEVFKNAPLDFKP

SEQ ID NO: 42 - protein, 103 aa, Klebsiella oxytoca
SPAKGSKQGQGGASQAEINSYISQVQAAIKGHLYDWDTYKGKTCTLRVNLAQDGTLLSVKSEGGDPAFCQQMLAATSRMTKFPKPPSQAVYETFKNAPLDFKP

SEQ ID NO: 43 - protein, 104 aa, Salmonella enterica
QPSKDSGTSGANGGATGADISAYAKQIQVAIQSRLYDASLYQGKQCVLHISLAPDGSLKSITSEGGDPALCQAALMAAKTAKIPKPPSQAVYEKIKDAKLDFKL

SEQ ID NO: 44 - protein, 104 aa, Salmonella typhimurium; identical to SEQ ID NO: 43

SEQ ID NO: 45 - protein, 104 aa, Salmonella typhi
QPSKDSGTSGANGGTTGADISAYAKQIQVAIQSRLYDASLYQGKQCVLHISLAPDGSLKSITSEGGDPALCQAALMATKTAKIPKPPSQAVYEKIKDAKLDFKL

SEQ ID NO: 46 - protein, 78 aa, Salmonella paratyphi
TVDLSSIADSALLSVAEKTIYADAQKSAILSVGPGMLREIKRADATGRQISTFEGDPAVTWAPFQSGKRQVTSFNNQA

SEQ ID NO: 47 - protein, 97 aa, Yersinia pestis
GGKKSGASGADISGYLGQITGAIQSKFYDADLYKGRTCDLRIKLAPDGLLIDVKAEGGDPALCQAAIAAAKQAKIPKPPSTDVYEQFKNAPLVFKPQ

SEQ ID NO: 48 - protein, 97 aa, Yersinia enterocolitica
GGGKKSGASAGDISGYLGQVTAAIQSKFYDADLYKGRTCNLRIKLAPDGLLIDVKQEGGDPALCQAAIAAAKLAKIPKPPSQDVYEVFKNAPLVFKP

SEQ ID NO: 49 - protein, 97 aa, Yersinia pseudotuberculosis
GGGKKSGASGADISGYLGQITGAIQSKFYDADLYKGRTCDLRIKLAPDGLLIDVKAEGGDPALCQAAIAAAKQAKIPKPPSTDVYEQFKNAPLVFKP

SEQ ID NO: 50 - protein, 97 aa, Acinetobacter baumannii
GNTAKHAGHVAADTTVKATKKTTGKVTELSSKAATGTKHVASEAVTGTKHFATEAATGTKNLATKAATGTKNLAVEAKADTKAHLDAVKTKVAEKQA

SEQ ID NO: 51 - protein, 102 aa, Acinetobacter calcoaceticus
QVTPTQVADNKIISEAEKYKKQYQKKIYKTWDIPKSSTGMSASVKVFLTDTGEIEQIIFLDKEEQKFKTSIEKAIWHSSPFVLPSNPEVRKQARKFNIKFQV

SEQ ID NO: 52 - protein, 103 aa, Stenotrophomonas maltophilia
QPAGGGGGTSDDLSAKYAAAIQAKVLSQWVRPDTVPLGQRCTITITQIPGGTVMQAKVSPNCPYDEAGRRSIEAAVLNAQPLPYRGFESVFARTLNFTFTAQD

SEQ ID NO: 53 - protein, 88 aa, Francisella tularensis
DLAVSKYVTEYTDRVGSNWIKDDCRGIYDLPRAIIRNGKFIRLTGTSGDYGCDQSLIDAIKNTTPPDISNPVARRTIQTENISFIFKQ

SEQ ID NO: 54 - protein, 142 aa, Escherichia coli str. K-12 substr. MG1655 chromosome
MARARGRGRRDLKSEINIVPLLDVLLVLLLIFMATAPIITQSVEVDLPDATESQAVSSNDNPPVIVEVSGIGQYTVVVEKDRLERLPPEQVVAEVSSRFKANPKTVFLIGGAKDVPYDEIIKALNLLHSAGVKSVGLMTQPI

SEQ ID NO: 55 - protein, 142 aa, Enterobacter cloacae EcWSU1
MARSRGRNRRELKSEINIVPLLDVLLVLLLIFMATAPIITQSVEVDLPDATESQAVSTNDDPPVIIEVSGVGQYSVVVEKDRMDQLPPEQVIAEAQRRLESNPKTVFLIGGAKDVPYDEIIKALNLLHSAGVKSVGLMTQPI

SEQ ID NO: 56 - protein, 142 aa, Shigella flexneri; identical to SEQ ID NO: 54

SEQ ID NO: 57 - protein, 142 aa, Shigella sonnei; identical to SEQ ID NO: 54

SEQ ID NO: 58 - protein, 142 aa, Shigella boydii
MARARGRGRRELKSEINIVPLLDVLLVLLLIFMATATIITQSVEVDLPDATESQAVSSNDNPPVIVEVSGIGQYTVVVEKDRLERLPPEQVVAEVSSRFKANPKTVFLIGGAKDVPYDEIIKALNLLHSAGVKSVGLMTQPI

SEQ ID NO: 59 - protein, 142 aa, Shigella dysenteriae; identical to SEQ ID NO: 54

SEQ ID NO: 60 - protein, 146 aa, Pseudomonas aeruginosa
MARVRHKRKPVAEMNVVPYIDVMLVLLVIFMVTAPMLNQGVKVDLPKVSSEALPQDNNKQVLTLSVKADGSYYWNVGSEVDTEKQTDSAVSLEQMTDAVTKIMSARPDTQVFIRGDKAVNYGAVVGAMGALQQAGVPNVGLITEAP

SEQ ID NO: 61 - protein, 128 aa, Pseudomonas putida
MQQREPSASESYFWPSVWAIGLHVLVFALLFVSFAMTPELPPSKPIVQATLYQLKSKSQATTQTNQKIAGEAKKTASRQTEVEQLEQKKVEQEAVKAAEQKKADAAQKAEEAREAAEAKKSRGCCQGR

SEQ ID NO: 62 - protein, 153 aa, Pseudomonas fluorescens
MALIARARKKRKPVAEMNVVPYIDVMLVLLVIFMVTAPMLNQGVKVDLPKVSSEALPQDNNTQVLTISIKADKTYYWNLGSEVDTEKQQDRAMTLPQMTDAVTKIIRVGNDAGKRTQVFIRGDKTVDYGAVMGAMGGLQKAGVGNVGLITEAP

SEQ ID NO: 63 - protein, 146 aa, Pseudomonas stutzeri
MARIRNRRKPVAEMNVVPYIDVMLVLLVIFMVTAPMLNQGVKVDLPKVSSEALPQDNDKQVLTISIKADKTYYWNVGSEVDTESKGNEAVALEDMTRAVVAIMRQRPDTQVFIRGDRAVDYGAVMAAMGGLQEAGVGNVGLITEAP

SEQ ID NO: 64 - protein, 142 aa, Klebsiella pneumoniae
MARARGRGRRELKSEINIVPLLDVLLVLLLIFMATAPIITQSVEVDLPDATESQAVKSNDDPPVIVEVSGVGQYSVKVGQETLSQLPPEQVIAEAKRRLEANEKTVFLIGGAKDVPYDEIIKALNLLHSAGVKSVGLMTKPI

SEQ ID NO: 65 - protein, 142 aa, Klebsiella oxytoca
MARARGRGRRELKSEINIVPLLDVLLVLLLIFMATAPIITQSVEVDLPDATESQAVKSNDEPPVIVEVSGVGQYSVKVGPETLSQLPPEQVIAEARRRLEANEKTVFLIGGAKEVPYDEIIKALNLLHSAGVKSVGLMTKPI

SEQ ID NO: 66 - protein, 142 aa, Salmonella enterica
MARTRGRGRRELKSEINIVPLLDVLLVLLLIFMATAPIITQSVEVDLPEANQSQAVSSNDDPPVIIEVSGVGQYSVVVDKDRMDQLPSEQVIAEVKRHLQANPKTVFLIGGAKEVPYDEIIKALNLLHSAGVKSVGLMTQPI

SEQ ID NO: 67 - protein, 142 aa, Salmonella typhimurium; identical to SEQ ID NO: 66

SEQ ID NO: 68 - protein, 142 aa, Salmonella typhi; identical to SEQ ID NO: 66

SEQ ID NO: 69 - protein, 142 aa, Salmonella paratyphi; identical to SEQ ID NO: 66

SEQ ID NO: 70 - protein, 142 aa, Yersinia pestis
MARVRGRKRRELKSEINIVPLLDVLLVLLLIFMATAPIITQSVEVNLPDATDSKTVSSDDNPPVIVEVSGVGQYTVVVDHQRMELLPSEQVVAEAQARLKTNPKTVFLIGGAKEVPYDEIIKALNMLHQAGVTSVGLMTQPI

SEQ ID NO: 71 - protein, 142 aa, Yersinia enterocolitica
MARVRGRKRRELKSEINIVPLLDVLLVLLLIFMATAPIITQSVEVDLPDATDSKTVSSDDNPPVIVEVSGVGQYSLVIDHQRMEQLPSEQVVAEAQARLKSNPKTVFLIGGAKEVPYDEIIKALNMLHQAGVTSVGLMTQPI

SEQ ID NO: 72 - protein, 142 aa, Yersinia pseudotuberculosis; identical to SEQ ID NO: 70

SEQ ID NO: 73 - protein, 150 aa, Acinetobacter baumannii
MAIQRSGRFERIKKPLKSDMNVVPYIDVMLVLLVIFMVTAPMITSGIKVDLPQANNHPIESKDMPAMVTIGKDGNIYLQYKDYKDNEPLSLEKLESVLSEAQKQAEQENKQLTVLVNGDKTRPYGEVVSLMASLQEAGLTQVGLLTEPLK

SEQ ID NO: 74 - protein, 150 aa, Acinetobacter calcoaceticus
MAIQRSGRFERIKKPLKSDMNVVPYIDVMLVLLVIFMVTAPMITSGIKVDLPQANNHPIESKDMPAMVTIGKDGNIYLQYEDYKDNEPLSREKLEEVLSDAQKQAEQENKQLTVLVNGDKSRPYGEVVSLMASLQEAGLTQVGLLTEPLK

SEQ ID NO: 75 - protein, 142 aa, Stenotrophomonas maltophilia
MSAAIGRRKRRKLKSEINVVPYIDVMLVLLIIFMVTAPLLTLSFDVDLPQSNAKALESKQDPVIVSVRQDGQLSLKLPDAKEPAAVSAEELEGRLAGIAAQDKGVRVIVAADRAVAYEKVIAAMDVIKRAKVDKVGLATDAR

SEQ ID NO: 76 - protein, 303 aa, Francisella tularensis
MANLNYNKFLRFCNKQIDENPFLVKAILIHIALIILLYILSFVSSLKFEKTQASLTAQVSNMPKKFEIIQATSISSSELNKQISAYENHQQELKQAKEDIKQAKLQALRKHQQQLKEKAEAEKKAKQQAILEAKKKAQQEAQRQAEQEKQAKLEAERKAKAEAEQKAQQELQRKKEQELKAKQQAEEKARQEKLAKARAEAEAAARKQIEQNQAQSAISSYIAAYQDRVGANWIKDSCRGIYDLPRAIIRDGKFIKLTGTSGNYRCDQSLIDAIKNTTPPTITNNVARKTIQTENISFIFKQS

SEQ ID NO: 77 - protein, 74 aa, Escherichia coli
EVDLPDATESQAVSSNDNPPVIVEVSGIGQYTVVVEKDRLERLPPEQVVAEVSSRFKANPKTVFLIGGAKDVPY

SEQ ID NO: 78 - protein, 74 aa, Enterobacter cloacae
EVDLPDATESQAVSTNDDPPVIIEVSGVGQYSVVVEKDRMDQLPPEQVIAEAQRRLESNPKTVFLIGGAKDVPY

SEQ ID NO: 79 - protein, 74 aa, Shigella flexneri; identical to SEQ ID NO: 77

SEQ ID NO: 80 - protein, 74 aa, Shigella sonnei; identical to SEQ ID NO: 77

SEQ ID NO: 81 - protein, 74 aa, Shigella boydii; identical to SEQ ID NO: 77

SEQ ID NO: 82 - protein, 74 aa, Shigella dysenteriae; identical to SEQ ID NO: 77

SEQ ID NO: 83 - protein, 80 aa, Pseudomonas aeruginosa
KVDLPKVSSEALPQDNNKQVLTLSVKADGSYYWNVGSEVDTEKQTDSAVSLEQMTDAVTKIMSARPDTQVFIRGDKAVNY

SEQ ID NO: 84 - protein, 67 aa, Pseudomonas putida
TPELPPSKPIVQATLYQLKSKSQATTQTNQKIAGEAKKTASRQTEVEQLEQKKVEQEAVKAAEQKKA

SEQ ID NO: 85 - protein, 84 aa, Pseudomonas fluorescens
KVDLPKVSSEALPQDNNTQVLTISIKADKTYYWNLGSEVDTEKQQDRAMTLPQMTDAVTKIIRVGNDAGKRTQVFIRGDKTVDY

SEQ ID NO: 86 - protein, 80 aa, Pseudomonas stutzeri
KVDLPKVSSEALPQDNDKQVLTISIKADKTYYWNVGSEVDTESKGNEAVALEDMTRAVVAIMRQRPDTQVFIRGDRAVDY

SEQ ID NO: 87 - protein, 74 aa, Klebsiella pneumoniae
EVDLPDATESQAVKSNDDPPVIVEVSGVGQYSVKVGQETLSQLPPEQVIAEAKRRLEANEKTVFLIGGAKDVPY

SEQ ID NO: 88 - protein, 74 aa, Klebsiella oxytoca
EVDLPDATESQAVKSNDEPPVIVEVSGVGQYSVKVGPETLSQLPPEQVIAEARRRLEANEKTVFLIGGAKEVPY

SEQ ID NO: 89 - protein, 74 aa, Salmonella enterica
EVDLPEANQSQAVSSNDDPPVIIEVSGVGQYSVVVDKDRMDQLPSEQVIAEVKRHLQANPKTVFLIGGAKEVPY

SEQ ID NO: 90 - protein, 74 aa, Salmonella typhimurium; identical to SEQ ID NO: 89

SEQ ID NO: 91 - protein, 74 aa, Salmonella typhi; identical to SEQ ID NO: 89

SEQ ID NO: 92 - protein, 74 aa, Salmonella paratyphi; identical to SEQ ID NO: 89

SEQ ID NO: 93 - protein, 74 aa, Yersinia pestis
EVNLPDATDSKTVSSDDNPPVIVEVSGVGQYTVVVDHQRMELLPSEQVVAEAQARLKTNPKTVFLIGGAKEVPY

SEQ ID NO: 94 - protein, 74 aa, Yersinia enterocolitica
EVDLPDATDSKTVSSDDNPPVIVEVSGVGQYSLVIDHQRMEQLPSEQVVAEAQARLKSNPKTVFLIGGAKEVPY

SEQ ID NO: 95 - protein, 74 aa, Yersinia pseudotuberculosis; identical to SEQ ID NO: 93

SEQ ID NO: 96 - protein, 77 aa, Acinetobacter baumannii
KVDLPQANNHPIESKDMPAMVTIGKDGNIYLQYKDYKDNEPLSLEKLESVLSEAQKQAEQENKQLTVLVNGDKTRPY

SEQ ID NO: 97 - protein, 77 aa, Acinetobacter calcoaceticus
KVDLPQANNHPIESKDMPAMVTIGKDGNIYLQYEDYKDNEPLSREKLEEVLSDAQKQAEQENKQLTVLVNGDKSRPY

SEQ ID NO: 98 - protein, 73 aa, Stenotrophomonas maltophilia
DVDLPQSNAKALESKQDPVIVSVRQDGQLSLKLPDAKEPAAVSAEELEGRLAGIAAQDKGVRVIVAADRAVAY

SEQ ID NO: 99 - protein, 110 aa, Francisella tularensis
QASLTAQVSNMPKKFEIIQATSISSSELNKQISAYENHQQELKQAKEDIKQAKLQALRKHQQQLKEKAEAEKKAKQQAILEAKKKAQQEAQRQAEQEKQAKLEAERKAKA

SEQ ID NO: 100 - protein, 79 aa, artificial sequence
  amino acid residues 29 to 107 of Colicin A
SHGNSGGHDRGDSSNVGNESVTVMKPGDSYNTPWGKVIINAAGQPTMNGTVMTADNSSMVPYGRGFTRVLNSLVNNPVS

SEQ ID NO: 101 - DNA, 235 bp, artificial sequence
  nucleid acid encoding amino acid residues 29 to 107 of Colicin A
ccatggaaatagtggtgggcacgatcgtggagattcttccaacgtaggtaatgagtctgtgacggtaatgaaaccaggggattcgtataacaccccgtggggaaaagtcatcatcaatgctgcaggccagccgaccatgaacggaacggtgatgaccgctgataattcatcgatggttccttacggcagagggtttacacgggttttaaattccctggtcaataatcctgtttcg

SEQ ID NO: 102 - protein, 81 aa, M13 phage
AETVESCLAKCMGCIEGRAHTENSFTNVWKDDKTLDRYANYEGCLWNATGVVVCTGDETQCYGTWVPIGLAIPENEGGGSE

SEQ ID NO: 103 - DNA, 243 bp, M13 phage
gctgaaactgttgaaagttgtttagccaaatgcatgggatgcatcgagggtcgcgcgcatacagagaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgag

SEQ ID NO: 104 - protein, 30 aa, artificial sequence
  One-Strep tag
SAWSHPQFEKGGGSGGGSGGSAWSHPQFEK